(19) United States
(12) Patent Application Publication
(10) Pub. No.: US 2025/0140339 A1
(43) Pub. Date: May 1, 2025

(54) GENETIC-BASED BIOLOGICAL SAMPLE ANSLYSIS SYSTEMS AND METHODS FOR DETECTING A USER-SPECIFIC GENETIC HEALTH RISK

(71) Applicant: Grifols S.A., Barcelona (ES)

(72) Inventors: Monica Lopez Martinez, Barcelona (ES); Marta Artieda Oseñalde, Barcelona (ES); Amaia Larruskain Mandiola, Barcelona (ES)

(21) Appl. No.: 18/384,711

(22) Filed: Oct. 27, 2023

(51) Int. Cl. G16B 20/20; G16B 40/20; G16H 10/40; G16H 50/30

(52) U.S. Cl. CPC G16B 20/20; G16B 40/20; G16H 10/40; G16H 50/30

(57) ABSTRACT

Genetic-based biological sample analysis systems and methods for detecting a user-specific genetic health risk related to a disease are disclosed herein. The systems and methods comprise obtaining user specific data of a user for detecting a likelihood of occurrence of a user-specific disease. Generation of a user profile causes a user test kit to be delivered to the user for collecting a biological sample comprising genomic deoxyribonucleic acid (DNA) of the user. Lab-based genetic analysis output, defining allele(s) of the user selected from related clinically relevant allelic variant genotype(s), is determined from the biological sample, and a classification is output by a genetic risk model defining a predetermined risk category based on the user's identified allele(s). A user-specific genetic health risk determination is generated based on the classification of the user and the lab-based genetic analysis output and is provided to the user for display on a user interface.

### FIELD

The present disclosure generally relates to genetic-based biological sample analysis systems and methods, and more particularly to, genetic-based biological sample analysis systems and methods for detecting a user-specific genetic health risk related to a disease, e.g., lung disease or liver disease.

### BACKGROUND

Individuals typically fail to test for individual genetic health risk, which can lead to life threating conditions. Such failures often result based on inconvenience, including the need to conduct such genetic health test at a remote location such as a doctor's office and under the prescription of a health care professional.

In addition, even if an individual does test for individual genetic health risk, a problem arises in accurately determining a specific risk type of a specific individual. This is at least because, in some instances, there are too few clinical cases associated with a given disease. In other instances, there may be numerous clinical cases associated with a given disease, but where such clinical cases lack sufficient consensus in order to provide a statistically significant genetic-based determination for the individual. In such cases, the individual can remain without a determination as his or her user-specific genetic health risk related to a disease and may have no way to determine whether to actively monitor such risks for possible future life threatening conditions.

Still further, existing genetic health risk tests often focus on over use of data and/or can be erroneous due to overlapping ranges and/or insufficient data with respect to clinical genetic testing. Such problems create false positives and/or false negative data and information, which cannot only be detrimental to the user, but also to the underlying computing system in the form of prediction accuracy and increased data storage of a user's specific health related information.

For the foregoing reasons, there is a need for genetic-based biological sample analysis systems and methods for detecting a user-specific genetic health risk related to a disease, e.g., lung disease, liver disease, or thrombophilia disease. There is also a need for such systems and methods that could be implemented not only at the doctor's office and under the prescription of a Health Care Professional, but also suitable for over-the-counter use.

### SUMMARY

Generally, as described herein, genetic-based biological sample analysis systems and methods for detecting a user-specific genetic health risk related to a disease, e.g., lung disease or liver disease. In various aspects, the genetic-based biological sample analysis systems and methods use qualitative genotyping to detect clinically relevant genetic variants associated with specific diseases. For example, in one example implementation, detection of one disease (e.g., .g., lung and/or liver disease) includes detection of clinically relevant variants associated with alpha-1 antitrypsin deficiency (AATD) in genomic DNA isolated from human salvia. Such analysis may facilitate the detection, identification, and reporting of user-specific data, including a genetic health risk determination for a given user. The determination may indicate, for example, that the given user is classified as having an increased risk of developing a specific disease, e.g., lung and/or liver disease as linked to AATD. Such determination may be based on identification of specific genetic variants of a user, including certain numbers thereof, including, for example, 14 possible genetic variants for AATD in the SERPINA1 gene, which may include by way of non-limiting example.: PI*S; PI*Z; PI*I, PI*M Procida; PI*M Malton; PI*S Iiyama; PI*Q0 granite falls; PI*Q0 west; PI*Q0 Bellingham; PI*F; PI*P Lowell; PI*Q0 Mattawa; PI*Q0 clayton, and PI*M Heerlen.

More particularly, as described herein, a genetic-based biological sample analysis system is disclosed for detecting a user-specific genetic health risk related to a disease. The genetic-based biological sample analysis system comprises a server comprising one or more processors and server computing instructions configured for execution by the one or more processors of the server, the server communicatively coupled to a computer network. The genetic-based biological sample analysis system may further comprise a genetic risk model stored in a memory communicatively coupled to the server and accessible by the one or more processors of the server. The genetic risk model may be configured to output a classification defining respective risks of respective users developing the disease. The classification as output by the genetic risk model may be selected from a predetermined set of risk categories comprising: (a) an Increased Risk Category, (b) a Slightly Increased Risk Category, (c) a Not Likely at Increased Risk Category, and (d) an Unknown Risk Category. The risk categories are ordinal with respect to one another ordered based on respective percentage risk values or ranges of contracting the disease, wherein the Increased Risk Category is assigned an upper percentage risk value or range, wherein the Not Likely at Increased Risk Category is assigned a lower percentage risk value or range, wherein the Slightly Increased Risk Category comprises a middle percentage risk value or range that is less than the upper percentage risk value or range but greater than the lower percentage risk value or range, and wherein the Unknown Risk Category has an undetermined percentage risk value or range. The genetic-based biological sample analysis system may further comprise a user application (app) implementing a user interface and comprising computing instructions configured for execution on a computing device. The user app may be configured to send to and receive data from the server. The server computing instructions, when executed by the one or more processors of the server, may cause the one or more processors of the server to obtain, from a display of the computing device, user specific data of a user for detecting a likelihood of occurrence of a user-specific disease. The server computing instructions, when executed by the one or more processors of the server, may further cause the one or more processors of the server to generate a profile of the user comprising the user specific data, wherein generation of the profile causes a user test kit to be delivered to the user, wherein the user test kit is configured to collect a biological sample of the user, the biological sample comprising genomic deoxyribonucleic acid (DNA) of the user as extracted from the biological sample. The server computing instructions, when executed by the one or more processors of the server, may further cause the one or more processors of the server to receive lab-based genetic analysis output based on the genomic DNA of the user, wherein generation of the lab-based genetic analysis output comprises determination of one or more alleles of the user selected from one or more clinically relevant allelic variant genotypes. The server computing instructions, when executed by the one or more processors of the server, may further cause the one or more processors of the server to output a classification for the user, based on the lab-based genetic analysis output, by the genetic risk model, the classification defining a user-specific risk of the user to develop the disease based on the one or more alleles as selected for the user. The server computing instructions, when executed by the one or more processors of the server, may further cause the one or more processors of the server to generate a user-specific genetic health risk determination for the user based on the classification of the user and the lab-based genetic analysis output. The server computing instructions, when executed by the one or more processors of the server, may further cause the one or more processors of the server to transmit, to the user app, the user-specific genetic health risk determination for display on the user interface.

In addition, as described herein, a genetic-based biological sample analysis method is disclosed for detecting a user-specific genetic health risk related to a disease. The genetic-based biological sample analysis method comprises obtaining, by one or more processors, user specific data of a user for detecting a likelihood of occurrence of a user-specific disease. The genetic-based biological sample analysis method may further comprise generating, by the one or more processors, a profile of the user comprising the user specific data, wherein generation of the profile causes a user test kit to be delivered to the user, wherein the user test kit is configured to collect a biological sample of the user, the biological sample comprising genomic deoxyribonucleic acid (DNA) of the user as extracted from the biological sample. The genetic-based biological sample analysis method may further comprise receiving, at the one or more processors, lab-based genetic analysis output based on the genomic DNA of the user, wherein generation of the lab-based genetic analysis output comprises determination of one or more alleles of the user selected from one or more clinically relevant allelic variant genotypes. The genetic-based biological sample analysis method may further comprise outputting, by the one or more processors, a classification for the user, based on the lab-based genetic analysis output, by a genetic risk model, the classification defining a user-specific risk of the user to develop the disease based on the one or more alleles as selected for the user, wherein the classification as output by the genetic risk model is selected from a predetermined set of risk categories comprising: (a) an Increased Risk Category, (b) a Slightly Increased Risk Category, (c) a Not Likely at Increased Risk Category, and (d) an Unknown Risk Category, wherein the genetic risk model is configured to output a classification defining respective risks of respective users developing the disease. The risk categories are ordinal with respect to one another ordered based on respective percentage risk values or ranges of contracting the disease, wherein the Increased Risk Category is assigned an upper percentage risk value or range, wherein the Not Likely at Increased Risk Category is assigned a lower percentage risk value or range, wherein the Slightly Increased Risk Category comprises a middle percentage risk value or range that is less than the upper percentage risk value or range but greater than the lower percentage risk value or range; wherein the Unknown Risk Category has an undetermined percentage risk value or range. The genetic-based biological sample analysis method may further comprise generating, by the one or more processors, a user-specific genetic health risk determination for the user based on the classification of the user and the lab-based genetic analysis output. The genetic-based biological sample analysis method may further comprise providing, by the one or more processors, to the user the user-specific genetic health risk determination.

Still further, as described herein, a tangible, non-transitory computer-readable medium is disclosed. The tangible, non-transitory computer-readable medium store instructions for detecting a user-specific genetic health risk related to a disease, that when executed by one or more processors, cause the one or more processors to obtain, by one or more processors, user specific data of a user for detecting a likelihood of occurrence of a user-specific disease. The instructions, when executed, may further cause the one or more processors to generate, by the one or more processors, a profile of the user comprising the user specific data, wherein generation of the profile causes a user test kit to be delivered to the user, wherein the user test kit is configured to collect a biological sample of the user, the biological sample comprising genomic deoxyribonucleic acid (DNA) of the user as extracted from the biological sample. The instructions, when executed, may further cause the one or more processors to receive, at the one or more processors, lab-based genetic analysis output based on the genomic DNA of the user, wherein generation of the lab-based genetic analysis output comprises determination of one or more alleles of the user selected from one or more clinically relevant allelic variant genotypes. The instructions, when executed, may further cause the one or more processors to output, by the one or more processors, a classification for the user, based on the lab-based genetic analysis output, by a genetic risk model, the classification defining a user-specific risk of the user to develop the disease based on the one or more alleles as selected for the user. The genetic risk model is configured to output a classification defining respective risks of respective users developing the disease, wherein the classification as output by the genetic risk model is selected from a predetermined set of risk categories comprising: (a) an Increased Risk Category, (b) a Slightly Increased Risk Category, (c) a Not Likely at Increased Risk Category, and (d) an Unknown Risk Category, wherein the risk categories are ordinal with respect to one another ordered based on respective percentage risk values or ranges of contracting the disease, wherein the Increased Risk Category is assigned an upper percentage risk value or range, wherein the Not Likely at Increased Risk Category is assigned a lower percentage risk value or range, wherein the Slightly Increased Risk Category comprises a middle percentage risk value or range that is less than the upper percentage risk value or range but greater than the lower percentage risk value or range, wherein the Unknown Risk Category has an undetermined percentage risk value or range. The instructions, when executed, may further cause the one or more processors to generate, by the one or more processors, a user-specific genetic health risk determination for the user based on the classification of the user and the lab-based genetic analysis output. The instructions, when executed, may further cause the one or more processors to provide, by the one or more processors, to the user the user-specific genetic health risk determination.

In accordance with the above, and with the disclosure herein, the present disclosure includes improvements in computer functionality or in improvements to other technologies at least because the claims recite, e.g., use of a genetic risk model which streamlines output and reduces storage of user specific data by implementing a classifier. That is, the genetic risk model inputs for analysis thereby a user's genomic DNA, as identified in a large set of data defined by a lab-based genetic analysis output and reduces such data into a classification output selected from predetermined set of risk categories. Such data reduction can reduce data memory needed for a specific user, and, even more so across an entire database system, which may store data for hundreds or thousands of users. Such implementation reduces data storage for the system as a whole, and, thereby improves it. Still further, by storing classification-based data, instead a full data stack, later operations performed on such data, such as generation of a user-specific genetic health risk determination requires reduced processing power of the underlying computing system, e.g., a server, given that the processors of the underlying computing system need operation on only a minimized dataset (e.g., one or more predetermined classifications or otherwise categories). This improves over the prior art at least because prior art systems require storage and processing of more fulsome datasets for each user, and, in some cases for each time a request to generate a specific report or determination for the user is made. Such prior art systems are comparatively inefficient, requiring more data storage and processing than the genetic-based biological sample analysis systems and methods disclosed herein.

In addition, the present disclosure relates to improvement to other technologies or technical fields at least because the classification or otherwise categories as output, and as determined based on a specific user's biological sample, reduce error output of the system as a whole, because the categories map to, or otherwise align to, clinically reported cases and provide output specific to the user with respect to specific disease risk. In addition, error is reduced by assigning an unknown risk category, which indicates that insufficient data exists and/or that overlapping or conflicting clinically reported cases relate to the user's identified biological sample (and related DNA). This reduces error by preventing both false positives and false negatives as output by prior art systems.

Still further, the present disclosure includes applying the certain of the claim elements with, or by use of, a particular machine, e.g., a server communicatively coupled to a Clinical Laboratory Improvement Amendments (CLIA) laboratory for generation and/or receipt of lab-based genetic analysis output based on the genomic DNA of a user.

Still further, the present disclosure includes effecting a transformation or reduction of a particular article to a different state or thing, e.g., effecting a transformation or reduction of a real-world biological sample (e.g., salvia of a user) comprising genomic deoxyribonucleic acid (DNA) into a user-specific genetic health risk determination by provision of lab-based genetic analysis output to a genetic risk model.

In addition, the present disclosure includes specific features other than what is well-understood, routine, conventional activity in the field, and that add unconventional steps that confine the claim to a particular useful application, e.g., genetic-based biological sample analysis systems and methods for detecting a user-specific genetic health risk related to a disease, e.g., lung disease or liver disease.

Advantages will become more apparent to those of ordinary skill in the art from the following description of the preferred aspects which have been shown and described by way of illustration. As will be realized, the present aspects may be capable of other and different aspects, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the system and methods disclosed therein. It should be understood that each Figure depicts a particular aspect of the disclosed system and methods, and that each of the Figures is intended to accord with a possible aspect thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present aspects are not limited to the precise arrangements and instrumentalities shown, wherein:

FIG. 1 illustrates an example genetic-based biological sample analysis system configured to detect a user-specific genetic health risk related to a disease, in accordance with various aspects disclosed herein.

FIG. 2 illustrates an example genetic-based biological sample analysis method for detecting a user-specific genetic health risk related to a disease, in accordance with various aspects disclosed herein.

FIG. 3 illustrates an example predetermined set of risk categories as may be output by a genetic risk model or otherwise classifier, in accordance with various aspects disclosed herein.

FIG. 4 illustrates an example graphic user interface (GUI) as rendered on a display screen of a user computing device depicting a user-specific genetic health risk determination of a user, in accordance with various aspects disclosed herein.

The Figures depict preferred aspects for purposes of illustration only. Alternative aspects of the systems and methods illustrated herein may be employed without departing from the principles of the invention described herein.

### DETAILED DESCRIPTION OF THE INVENTION

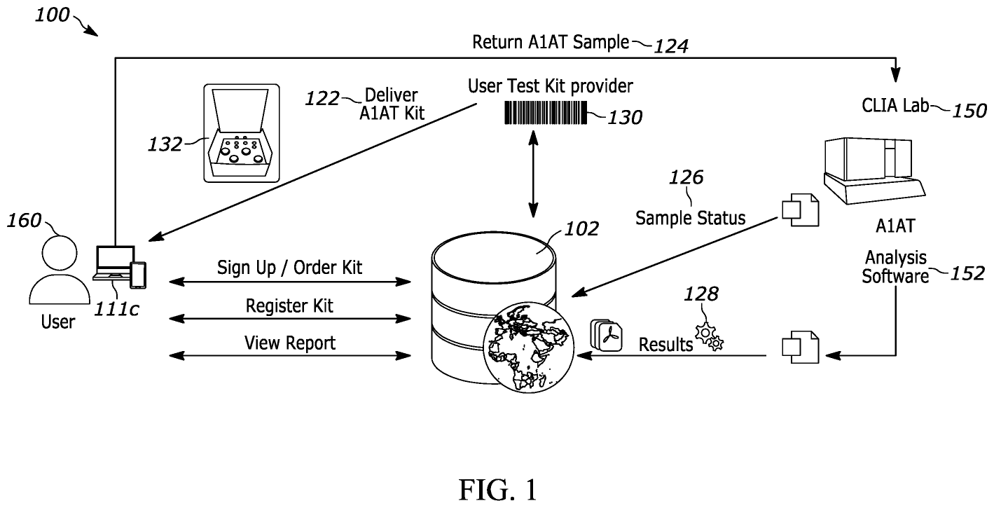

FIG. 1 illustrates an example genetic-based biological sample analysis system 100 configured to detect a user-specific genetic health risk related to a disease, in accordance with various aspects disclosed herein. In the example aspect of FIG. 1, genetic-based biological sample analysis system 100 includes a server 102, which may comprise one or more computer servers. In various aspects server 102 comprises multiple servers, which may comprise multiple, redundant, or replicated servers as part of a server farm. In still further aspects, server 102 may be implemented as cloud-based servers, such as a cloud-based computing platform. For example, server 102 may be any one or more cloud-based platform(s) such as MICROSOFT AZURE, AMAZON AWS, or the like. Server 102 may include one or more processor(s) as well as one or more computer memories. The processor(s) may be connected to the memories via a computer bus responsible for transmitting electronic data, data packets, or otherwise electronic signals to and from the processor(s) and memorie(s) in order to implement or perform the machine-readable instructions, methods, processes, elements or limitations, as illustrated, depicted, or described for the various flowcharts, illustrations, diagrams, figures, and/or other disclosure herein.

Server 102 may comprise computing instructions (e.g., which may be referred to herein, in at least some instances, as server computing instructions) stored on an accessible memory (e.g., one or more memories of the server accessible by the one or more processor(s) of the server). The one or more memories of server 102 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The one or more memories of server 102 may store an operating system (OS) (e.g., Microsoft Windows, Linux, UNIX, etc.) capable of facilitating the functionalities, apps, methods, or other software as discussed herein. In general, a computer program or computer based product, application, or code (e.g., an app or computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) (e.g., working in connection with the respective operating system in memories) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in a program language, and may be implemented as machine code, assembly code, byte code, interpretable source code or the like (e.g., via Golang, Python, C, C++, C#, Objective-C, Java, Scala, ActionScript, JavaScript, HTML, CSS, XML, etc.).

Additionally, or alternatively, data, user data, etc., may also be stored in the one or more memories and/or in a database, which may be accessible or otherwise communicatively coupled to server 102. In addition, memories of server 102 may also store machine readable instructions, including any of one or more application(s) (e.g., a genetic risk model and/or related computing instructions herein), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. For example, processor(s) of server 102 may access memory via the computer bus to execute an operating system (OS). Processor(s) of server 102 may also access the memory 106 via the computer bus to create, read, update, delete, or otherwise access or interact with the data stored in memories 106 and/or the database 105 (e.g., a relational database, such as Oracle, DB2, MySQL, or a NoSQL based database, such as MongoDB). The data, data structure, or other information stored in the one or more memories or databased accessible by server 102 may include all or part of any of the data or information described herein, including, for example, a genetic risk model.

With further reference to FIG. 1, the genetic-based biological sample analysis system 100 may further comprise a genetic risk model. In various aspects, the genetic risk model may be stored in a memory communicatively coupled to the server 102. The genetic risk model may be accessible by the one or more processors of the server 102. In various aspects, the genetic risk model comprises a set of computing instructions having, for example, an application programming interface (API) comprising an accessible set of computing functions for receiving input (e.g., genomic DNA of a user defining data of one or more alleles of a user selected from one or more clinically relevant allelic variant genotypes) and generating output, e.g., a given risk category as described herein, based on the input. Additionally, or alternatively, the genetic risk model may comprise a data structure, e.g., comprising a linked list or lookup table, accessible by processor of server 102 in order to lookup or compare risk categories based on a given user's genomic DNA.

In various aspects, the genetic risk model is configured to output, e.g., from an API of the genetic risk model, a classification defining respective risks of respective users developing the disease. The classification as output by the genetic risk model may be selected from a predetermined set of risk categories. For example, in various aspects, the risk categories may comprise a predetermined set of risk categories including: (a) an Increased Risk Category, (b) a Slightly Increased Risk Category, (c) a Not Likely At Increased Risk Category, and (d) an Unknown Risk Category. Each of the risk categories may be programmed into, or otherwise stored by, the genetic risk model, e.g., on a memory of server 102, for access, classification, and/or otherwise output, e.g., as described herein.

In various aspects, the risk categories may be ordinal with respect to one another. The risk categories may be stored in memory, linked in memory, or otherwise as part of genetic risk model in an ordinal manner. For example, the risk categories may be ordered with respect to one another, e.g., in memory, based on respective percentage risk values or ranges of contracting the disease. In one implementation, for example, one mode of ordinality comprises where the Increased Risk Category is assigned an upper percentage risk value or range (e.g., 80 percent) and the not Likely At Increased Risk Category is assigned a lower percentage risk value or range (e.g., 20 percent). Still further, the Slightly Increased Risk Category may comprise a middle percentage risk value or range (e.g., 20 percent to 80 percent) that is less than the upper percentage risk value or range but greater than the lower percentage risk value or range.

Still further, the Unknown Risk Category can have an undetermined percentage risk value or range. In various aspects, the Unknown Risk Category is a safety risk prediction or classification generated, for example, when there is insufficient data to detect a given disease for a specific user. For example, the Unknown Risk Category may be generated or output, e.g., by the genetic risk model, when there is insufficient information for a specific user for detecting lung disease or liver disease linked to AATD. As a further example, the Unknown Risk Category may be generated or output, e.g., by the genetic risk model, for genotypes having less than three reported clinical cases. In various aspects, insufficient information, e.g., a lack of reported clinical cases, may cause the user-specific genetic health risk determination risk, as generated for a user, to report or be displayed to the user that a risk developing a disease (e.g., lung or liver disease linked to AATD) is not known due to the lack of reported clinical cases or inconclusive data.

As a still further example, the Unknown Risk Category may be generated or output, e.g., by the genetic risk model, for those genotypes having three or greater reported clinical cases but with a calculated risk confidence interval overlapping with at least two other risk categories, where a first risk interval (measured at a 95% confidence interval) for Increased Risk Category (e.g., a first risk interval of 60% to 90%) overlaps with a second risk interval (measured at a 95% confidence interval) Slightly Increased Risk Category (e.g., a second risk interval of 30% to 70%). In various aspects, overlapping risk intervals may cause the user-specific genetic health risk determination risk, as generated for a user, to report or display to the user that a risk developing a disease (e.g., lung or liver disease linked to AATD) is unknown and that additional clinical studies are needed to determine the user's specific risk level.

With further reference to FIG. 1, server 102 may be coupled to a computer network (e.g., the Internet) for transmission of data or information thereon, for example, to facilitate user registration and to provide a user-specific genetic health risk determination. In various aspects, server 102 may include, or interact with, one or more transceivers (e.g., WWAN, WLAN, and/or WPAN transceivers) functioning in accordance with IEEE standards, 3GPP standards, or other standards, and that may be used in receipt and transmission of data via external/network ports connected to the computer network. In some aspects, computer network may comprise a private network or local area network (LAN). Additionally, or alternatively, computer network may comprise a public network such as the Internet.

In some aspects, server 102 may perform the functionalities as discussed herein as part of a “cloud” network or may otherwise communicate with other hardware or software components within the cloud to send, retrieve, or otherwise analyze data or information described herein. Server 102 may further include a communication component configured to communicate (e.g., send and receive) data via one or more external/network port(s) to one or more networks or local terminals, such as computer network for sending and receiving data or other information as described herein. In some aspects, server 102 may include a client-server platform technology such as ASP.NET, Java J2EE, Ruby on Rails, Node.js, a web service or online API, responsive for receiving and responding to electronic requests.

With further reference to FIG. 1, server 102 may be communicatively connected, via computer network 120, to the one or more user computing devices (e.g., computing device 111c) via a network connection such as a base station. In one aspects, for example, a base station may comprise comprises a cellular base station, such as a cell tower, communicating to the one or more user computing devices (e.g., computing device 111c) via wireless communications based on any one or more of various mobile phone standards, including NMT, GSM, CDMA, UMMTS, LTE, 5G, or the like. Additionally, or alternatively, a base station may comprise routers, wireless switches, or other such wireless connection points communicating to the one or more user computing devices (e.g., computing device 111c) via wireless communications 122 based on any one or more of various wireless standards, including by non-limiting example, IEEE 802.11a/b/c/g (WIFI), the BLUETOOTH standard, or the like.

As shown for FIG. 1, the one or more computing devices 111c (e.g., computing device 111c) may comprise mobile devices and/or client devices for accessing and/or communications with server 102. Such mobile devices may comprise one or more mobile processor(s). In various aspects, a user computing devices (e.g., computing device 111c) may comprise a mobile phone (e.g., a cellular phone), a tablet device, a personal data assistance (PDA), or the like, including, by non-limiting example, an APPLE IPHONE or IPAD device or a GOOGLE ANDROID based mobile phone or tablet. Additionally, in various aspects, an electronic device and/or display device, as described herein, may comprise a mobile phone (e.g., a cellular phone), a tablet device, a personal data assistance (PDA), or the like, including, by non-limiting example, an APPLE IPHONE or IPAD device or a GOOGLE ANDROID based mobile phone or tablet.

In various aspects, the one or more user computing devices (e.g., computing device 111c) may implement or execute an operating system (OS) or mobile platform such as APPLE iOS and/or Google ANDROID operation system. Any of the one or more user computing devices (e.g., computing device 111c) may comprise one or more processors and/or one or more memories for storing, implementing, or executing computing instructions or code, e.g., a mobile application, as described in various aspects herein. For example, a user computing device (e.g., user computing device 111c) may store locally on a memory of a user computing device a user application (app). In various aspects, the user app may implement a user interface (e.g., a GUI) and comprise computing instructions configured for execution on a computing device. The user app may be programmed, or otherwise configured, to send to and receive data from server 102. The user app may comprise a native application as implemented on a native operation system of the mobile device. Additionally, or alternatively, the user app may comprise a web browser application implemented, for example, via web programming languages or scripts, such as HTML and/or JavaScript, and accessed via web browser such as the GOOGLE CHROME web browser, the APPLE SAFARI web browser, or the like.

User computing devices (e.g., user computing device 111c) may comprise a wireless transceiver to receive and transmit wireless communications to and from server 102. In various aspects, data (e.g., such as user specific data and/or user-specific health risk determinations) may be transmitted via a computer network to server 102, as indicated for FIG. 1.

Still further, each of the one or more user computer devices (e.g., computing device 111c) may include a display screen for displaying graphics, images, text, instruction(s), data, and/or other such visualizations or information as described herein. In various aspects, graphics, images, text, instruction(s), data, and/or other such visualizations or information may be received from server 102 for display on the display screen of any one or more of user computer devices 111c. Additionally, or alternatively, a user computing device (e.g., computing device 111c) may implement, have access to, render, or otherwise expose, at least in part, an interface or a graphic user interface (GUI) for such graphics, images, text, instruction(s), data, and/or other such visualizations or information (e.g., such as a user-specific genetic health risk determination or report) on its display screen.

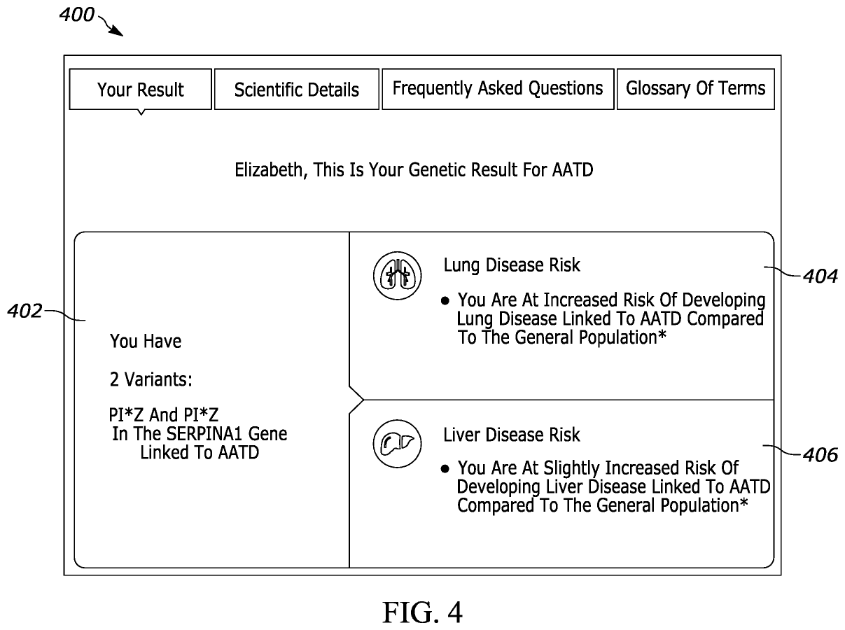

In various aspects, a display screen may also be used for providing information, instructions, and/or guidance to the user of a given device (e.g., user computing device 111c). For example, a display screen may be used to provide a user with a user-specific genetic health risk determination as described herein for FIG. 4. Additionally, or alternatively, a display screen may be configured to enable users to provide information (e.g., user specific data) to the server 102, which may be obtained, for example, by the user app implemented on computing device. For example, a user may provide information to the server 102 via inputting information into the display screen of user computing device 111c.

With reference to FIG. 1, user 160 may provide, from a display of a computing device (e.g., user computing device 111c) user specific data of the user, which may include the user's name, address, or other information for ordering a user test kit for detecting a likelihood of occurrence of a user-specific disease. Provision of the user specific data may cause server 102 to generate a profile for the user, where the profile may comprise the user specific data. Generation of the profile may cause a user test kit 132 to be delivered (122) to the user. The user test kit may be provided by a user test kit provider 130, which may receive information regarding user including the user specific data (e.g., address information) for shipping the user test kit to the user. In the example of FIG. 1, the user test kit is a A1AT based kit, but it should be understood that other test kits associated for testing different diseases or related genes are also contemplated herein.

In various aspects, user test kit 132 comprises is configured to collect a biological sample (e.g., saliva data) of the user. The biological sample may comprise saliva, hair, skin, or other such biological sample comprising genomic deoxyribonucleic acid (DNA) of the user as may be extracted from the biological sample. The user test kit 132 may comprise a container, which may include a preservative, for stabilizing or preserving the biological sample of the user, e.g., during transit. User test kit 132 may further comprise a unique identifier (e.g., a UPC code) that may be linked to the profile of the user, and which may be used to link the biological sample of the user to the user's data (e.g., genomic DNA).

In various aspects, user 160 may return (124) the biological sample, e.g., via a shipment service. As shown in FIG. 1, in some aspects, the biological sample is delivered to a laboratory, e.g., a Clinical Laboratory Improvement Amendments (CLIA) laboratory such as CLIA lab 150. CLIA lab 150 can extract genomic DNA of the user from the biological sample.

In various aspects, the genomic DNA may be analyzed by DNA detection software (e.g., A1AT analysis software 152) to determine one or more alleles of the user and/or one or more clinically relevant allelic variant genotypes of the user. The results 128 of such analysis may be provided, e.g., via computer network, to server 102 in the form of data or files. The results 128 may comprise lab-based genetic analysis output based on the genomic DNA of the user. The lab-based genetic analysis output may comprise a determination of one or more alleles of the user selected from one or more clinically relevant allelic variant genotypes. Such data or files may be stored on a memory of server 102. In addition, the CLIA lab (e.g., CLIA lab 150) may provide a sample status 126 as to analysis of a biological sample of the user. The sample status 126 may indicate a current progress or competition of analysis of the biological sample of the user.

Server 102 may input the results 128, including data regarding one or more alleles of the user selected from one or more clinically relevant allelic variant genotypes, into the genetic risk model as stored or otherwise accessible by server 102 and/or otherwise analyze the results by one or more processors of server 102. Server 102 may output a classification for the user based on the results 128 (e.g., lab-based genetic analysis output). The classification may be output by the genetic risk model. The classification may define a user-specific risk of the user 160 to develop the disease based on the one or more alleles as selected for the user, e.g., as described herein. In various aspects, user classification and/or the lab-based genetic analysis output may be analyzed by a processor of server 102 to generate a user-specific genetic health risk determination for the user. The user-specific genetic health risk determination may be transmitted to the user app, as implemented on user computing device 111c, for display on a user interface of the user computing device 111c, for example, as shown and described for FIG. 4 herein.

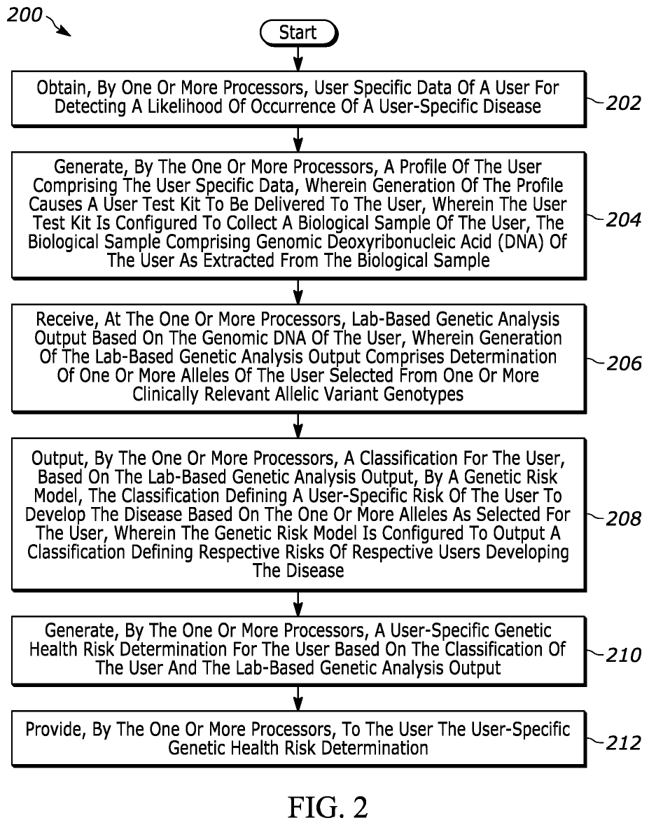

FIG. 2 illustrates an example genetic-based biological sample analysis method 200 for detecting a user-specific genetic health risk related to a disease, in accordance with various aspects disclosed herein. At block 202, genetic-based biological sample analysis method 200 comprises obtaining, by one or more processors, user specific data of a user for detecting a likelihood of occurrence of a user-specific disease. For example, a user may provide via an application (app), e.g., as describe herein for FIG. 1 on the user computing device 111c, a selection to determine their risk of developing a specific disease. The user may provide information such as the disease they want to be tested for, their age, their sex, whether they are a smoker, etc.

At block 204, genetic-based biological sample analysis method 200 comprises generating, by the one or more processors (e.g., one or more processors of server 102), a profile of the user comprising the user specific data, Generation of the profile causes a user test kit (e.g., user test kit 132) to be delivered to the user, such as a home address of the user as provided or indicated by the user. In various aspects, the user test kit may be configured, or may otherwise include equipment, to collect a biological sample (e.g., saliva, hair, skin, or otherwise) of the user. More generally, the biological sample comprises genomic deoxyribonucleic acid (DNA) of the user as extracted from the biological sample. For example, a generated profile of a user, e.g., based on information provided by the user, may comprise the disease the user wants to be tested for (e.g., alpha1-antitrypsin deficiency (A1ATD) associated with lung disease, A1ATD associated with liver disease), the user's age (e.g., 35 years old), the user's sex (e.g., female), and whether the user has ever smoked (e.g., nonsmoking). Upon generation of the profile, the user may receive a test kit configured to collect the saliva of the user. The saliva may comprise genomic DNA, which may be extracted from the saliva. It is to be understood that the invention may comprise kits that may collect DNA samples for other, different, or additional respective genes and/or related diseases.

At block 206, genetic-based biological sample analysis method 200 comprises receiving, at the one or more processors (e.g., one or more processors of server 102), lab-based genetic analysis output based on the genomic DNA of the user, wherein generation of the lab-based genetic analysis output comprises determination of one or more alleles of the user selected from one or more clinically relevant allelic variant genotypes. For example, genomic DNA extracted from saliva of the user may be amplified and biotinylated by multiplex PCR. The PCR products may be denatured and hybridized to oligonucleotide probes coupled to a color-coded bead. The oligonucleotide probes may be allele-specific oligonucleotide probes configured for one or more allelic variant genotypes clinically relevant to a disease, such as lung disease and/or liver disease as associated with A1AT deficiency. The hybridized PCR products (hybridized DNA) may be labeled with a fluorescent conjugate and the resulting signal may be detected to generate raw fluorescence data. The resulting signal may be detected by, e.g., a Luminex® 200™ system. Raw fluorescence data may be processed by software (e.g., analysis software 152) to determine DNA sequence data and/or one or more clinically relevant allelic variant genotypes identified in one or more alleles of the user. For example, the one or more alleles may be identified in the DNA sequence of data for the user. For example, analysis software 152 may be proprietary A1AT Genotyping Test Analysis Software configured to provide allelic variant genotypes, which are subsequently converted into one or more clinically relevant allelic variant genotypes associated with A1AT deficiency. As a further example, analysis software 152 may convert the allelic variant genotypes into associated alleles. Additionally, analysis software 152 may provide an associated template number for each sample, this is, a code that defines the type of user-specific genetic health risk determination (e.g., a genetic health risk report) that is to be generated for each individual, depending on a given individual's combination of variants. This template number can be subsequently used as a basis for the generation of personalized reports by the server 102. In this way, the analysis software 152 can generate a report with a codification of one or more (e.g., 121 possible) type of possible genotype combinations and replated (e.g., templates 1 to 121 as shown and described for Table 4 herein). In this way, a user-specific genetic health risk determination be converted or otherwise translated from a given template number or otherwise genotype combination into a report or otherwise determination specific to the user.

In some aspects, server 102 may subsequently receive from CLIA lab 150 and/or generate, at least in part, a lab-based genetic analysis output comprising the DNA sequence data and/or the one or more clinically relevant allelic variant genotypes identified in one or more alleles of the user. For example, the lab-based genetic analysis output comprising the DNA sequence data and/or the one or more clinically relevant allelic variant genotypes identified in one or more alleles of the user may be generated at least in part at server 102 and at least in part by CLIA lab 150 and/or analysis software 152.

In some aspects, the lab-based genetic analysis output further comprises one or more of: (a) a quantity of clinically reported cases of the respective one or more alleles, (b) a percentage of clinically reported cases of the respective one or more alleles in which the disease occurred, and/or (c) a respective risk-based confidence interval. For example, CLIA lab 150, analysis software 152, and/or server 102 may generate a lab-based genetic analysis output comprising current scientific evidence information, such as (i) the number of cases (times) the respective one or more alleles of the user have been reported (e.g., in published research articles), (ii) the percentage of cases in which the individual (e.g., user, patient) with the respective one or more alleles developed the disease associated with the respective one or more alleles, or (iii) the respective risk-based confidence interval of respective one or more alleles.

The aforementioned current scientific evidence information, and data thereof, may be stored on a memory of server 102, an external database or server communicatively coupled to server 102, which may comprise, for example, a database of CLIA lab 150 and/or of analysis software 152). In other aspects, such external server and/or database may comprise a publicly available server, which may comprise a server and/or database of the National Center for Biotechnology Information (NCBI)). In such aspects, server 102, CLIA lab 150 and/or of analysis software 152 may access current scientific evidence information and/or data to generate the lab-based genetic analysis output. Additionally, or alternatively, an administrator of the system 100 may upload current scientific evidence information or otherwise data to server 102, CLIA lab 150, and/or of analysis software 152 via an input/output (I/O) port communicatively coupled to server 102. Additionally, or alternatively, the current scientific evidence information or otherwise data may comprise one or more alleles of other users (e.g., users utilizing the system 100 previous to the current user) and/or one or more user profiles of other users. For example, one or more alleles of a previous user may be included in the quantity of clinically reported cases of the respective one or more alleles of the user (e.g., user 160) when one or more alleles of the previous user and the current user comprise the same one or more clinically relevant allelic variant genotypes.

In some aspects, the one or more alleles of the user may be selected from one or more clinically relevant allelic variants of the serpin peptidase inhibitor class A member 1 (SERPINA1) gene and the disease is lung disease and/or liver disease as associated with alpha1-antitrypsin deficiency (A1ATD).

In additional aspects, the one or more variants of the SERPINA1 gene include one or more of: (i) PI*S; (ii) PI*Z; (iii) PI*I; (iv) PI*M procida; (v) PI*M malton; (vi) PI*S iiyama; (vii) PI*Q0 granite falls; (viii) PI*Q0 west; (ix) PI*Q0 bellingham; (x) PI*F; (xi) PI*P lowell; (xii) PI*Q0 mattawa; (xiii) PI*Q0 clayton, and (xiv) PI*M Heerlen.

In additional aspects, the one or more clinically relevant allelic variant genotypes comprises one or more variants of a gene, wherein the gene is a Coagulation Factor V (FV) gene, Coagulation Factor II (FII) gene, Serpin family C member 1 (SERPINC1) gene, protein S (PROS1) gene, or protein C (PROC) gene; and wherein the disease is a thrombophilia associated disease.

In still further aspects, the one or more clinically relevant allelic variants is selected from the group consisting of: (i) R506Q of the FV gene, (ii) FV Leiden of the FV gene, (iii) G20210A of the FII gene, (iv) A384S (G1246T) (rs121909548) of the SERPINC1 gene, (v) Intronic (rs2227589) of the SERPIN1C gene, (vi) Val30Glu (rs2227624) of the SERPIN1C gene, (vii) K196E of the PROS1 gene, (vii) K155E of the PROS1 gene, (viii) c.574_576dup/del (rs199469469) of the PROC gene, (ix) R147W of the PROC gene, (x) Arg42Cys of the PROC gene, (xi) Arg51Cys of the PROC gene, (xii) Val76Met of the PROC gene, (xiii) C 2633 G of the PROC gene, (xiv) C 2730 T of the PROC gene, (xv) G 3310 A of the PROC gene, (xvi) c.565C.T (p.Arg189Trp) of the PROC gene, (xvii) 2405C/T of the PROC gene, (xviii) 2418A/G of the PROC gene, (xix) C/T at −1654, A/G at −1641, A/T at −1476 of the PROC gene, and (xx) promoter GC haplotype of the PROC gene.

With further reference to FIG. 2, at block 208, genetic-based biological sample analysis method 200 comprises outputting, by the one or more processors (e.g., one or more processors of server 102), a classification for the user. The classification may be output based on the lab-based genetic analysis output by a genetic risk model. The classification may define a user-specific risk of the user to develop the disease based on the one or more alleles as selected for the user. The genetic risk model is configured to output a classification defining respective risks of respective users developing the disease. In various aspects, the classification as output by the genetic risk model is selected from a predetermined set of risk categories comprising. In one example, the predetermined set of risk categories comprises (a) an Increased Risk Category, (b) a Slightly Increased Risk Category, (c) a Not Likely at Increased Risk Category, and (d) an Unknown Risk Category.

In various aspects, the risk categories are ordinal with respect to one another. For example, the risk categories may be ordered based on respective percentage risk values and/or ranges of contracting the disease. For example, the Increased Risk Category may be assigned an upper percentage risk value or range (e.g., 80 percent). The Not Likely at Increased Risk Category may be assigned a lower percentage risk value or range (e.g., 20 percent). Still further, the Slightly Increased Risk Category may comprise a middle percentage risk value or range (e.g., 20 percent to 80 percent) that is less than the upper percentage risk value or range but greater than the lower percentage risk value or range. Still further, the Unknown Risk Category has an undetermined percentage risk value or range.

For example, in some implementations, the classification as output for the user comprises the Unknown Risk Category. In such implementations, the outputted classification may be based upon detecting at least one of the following conditions, e.g., as further described herein for any one or more of Tables 1-7: (i) the one or more clinically relevant allelic variant genotypes as selected for the user are associated with two or less clinically reported cases, or (ii) the one or more clinically relevant allelic variant genotypes as selected for the user are associated with three or more clinically reported cases but the undetermined percentage risk value or range overlaps with at least one of the upper percentage risk value or range, the middle percentage risk value or range, or the lower percentage risk value or range.

In a still further example, in some implementations, the classification as output for the user comprises the Increased Risk category. In such implementations, the classification output may be based upon detecting that the one or more clinically relevant allelic variant genotypes as selected for the user are associated with three or more clinically reported cases, and where the upper percentage risk value or range is more than a minimum percent (e.g., 80%) as defined by clinically reported cases of the respective one or more alleles associated with an increased risk of developing the disease, and for a respective risk-based confidence interval. By way of non-limiting example with respect to lung disease linked to AATD, a minimum percent as defined by clinically reported cases of the respective one or more alleles characterized as an increased risk of developing lung disease may be equal to or approximately equal to 80%. In this example, such value can be based on a given allele and/or genotype (e.g., PI*Z/PI*Z) where a percentage of reported clinical cases with lung disease for users having the allele is 81.50% for respective risk-based confidence interval (95% confidence) with a lower bound of 80.40% and an upper bound of 82.60%. In such example, the lower bound value (e.g., 80.40%, which is approximately equal to 80%) represents the minimum percent as defined by clinically reported cases of the respective one or more alleles characterized as an increased risk of developing lung disease. Further values and ranges that may be used for additional and/or different implementations are described herein with respect to Tables 1-7.

In a still further example, in some implementations, the classification as output for the user comprises the Not Likely at Increased Risk category. In such implementations, the classification output may be based upon detecting that the one or more clinically relevant allelic variant genotypes as selected for the user are associated with three or more clinically reported cases, wherein the lower percentage risk value or range is at least one of: (i) less than a maximum percent as defined by clinically reported cases of the respective one or more alleles associated with a not likely increased risk of developing the disease, and for a respective risk-based confidence interval (e.g., a risk-based confidence interval between 0% to 80% at a 95% confidence), or (ii) between the maximum percent as defined by clinically reported cases of the respective one or more alleles associated with the not likely increased risk of developing the disease and a minimum percent as defined the clinically reported cases of the respective one or more alleles associated with an increased risk of developing the disease, and for such respective risk-based confidence interval (e.g., a risk-based confidence interval between 0% to 80% at a 95% confidence). By way of non-limiting example with respect to lung disease linked to AATD, a maximum percent as defined by clinically reported cases of the respective one or more alleles characterized as a not likely at risk of developing hng disease may be equal to or approximately equal to 20%. In this example, such value can be based on a given allele (e.g., PI*S/PI*S) where a percentage of reported clinical cases with lung disease for users having the allele is 8.70% for respective risk-based confidence interval (e.g., a risk-based confidence interval between 0% to 80% at a 95% confidence) with a lower bound of 4.00% and an upper bound of 18.00%. In such example, the upper bound value (e.g. 18.0%, which is approximately equal to 20%) represents the maximum percent as defined by clinically reported cases of the respective one or more alleles characterized as a not likely increased risk of developing the disease. In an additional example, two different confidence interval bounds of two different risk categories may be used for defining the Not Likely at Increased Risk category, where the maximum percent as defined by clinically reported cases of the respective one or more alleles associated with the not likely increased risk of developing the disease (e.g., 20% as described above for the Not Likely at Increased Risk category as associated with PI*S/PI*S) and a minimum percent as defined by the clinically reported cases of the respective one or more alleles associated with an increased risk of developing the disease (e.g., 80% as described above for the Increased Risk category as associated with PI*Z/PI*Z) are used, and where the middle percentage risk value or range is defined between these two values of 20% and 80%, respectively. Additionally, or alternatively, for further implementations may be used, including other or different confidence interval bounds of two different risk categories for defining the Not Likely at Increased Risk category. This includes, by way of non-limiting example, where the clinically reported cases identifying the disease are less than 20% but where the risk-based confidence interval of 95% for these cases has a value or range between 0-80%. As another example, where the clinically reported cases identifying the disease are between 20-80% but where the risk-based confidence interval of 95% for these cases has a value or range between 0-80%. Further values and ranges that may be used for additional and/or different implementations are described herein with respect to Tables 1-7.

In a still further example, in some implementations, the classification as output for the user comprises the Slightly Increased Risk category. In such implementations, the classification output may be based upon detecting that the one or more clinically relevant allelic variant genotypes as selected for the user are associated with three or more clinically reported cases, wherein the middle percentage risk value or range is at least one of: (i) more than a minimum percent as defined by clinically reported cases of the respective one or more alleles associated with an increased risk of developing the disease, and for a respective risk-based confidence interval (e.g., a risk-based confidence interval between 20% to 100% at a 95% confidence), or (ii) between a maximum percent as defined by clinically reported cases of the respective one or more alleles associated with the not likely increased risk of developing the disease and the minimum percent as defined the clinically reported cases of the respective one or more alleles associated with an increased risk of developing the disease, and for such respective risk-based confidence interval (e.g., a risk-based confidence interval between 20% to 100% at a 95% confidence). By way of non-limiting example with respect to lung disease linked to AATD, a minimum percent as defined by clinically reported cases of the respective one or more alleles characterized as an increased risk of developing lung disease may be equal to or approximately equal to 80% (as described above herein). In this example, such value can be based on a given allele (e.g., PI*Z/PI*Z) where a percentage of reported clinical cases with lung disease for users having the allele is 81.50% for respective risk-based confidence interval (e.g., a risk-based confidence interval between 20% to 100% at a 95% confidence) with a lower bound of 80.40% and an upper bound of 82.60%, as described above herein. In an additional example, two different confidence interval bounds of two different risk categories may be used for defining the Slightly Increased Risk category, where a maximum percent as defined by clinically reported cases of the respective one or more alleles associated with the not likely increased risk of developing the disease (e.g., 20% as described above for the Not Likely at Increased Risk category as associated with PI*S/PI*S) and a minimum percent as defined by the clinically reported cases of the respective one or more alleles associated with an increased risk of developing the disease (e.g., 80% as described above for the Increased Risk category as associated with PI*Z/PI*Z) are used, and where the middle percentage risk value or range is defined between these two values of 20% and 80%, respectively. Additionally, or alternatively, for further implementations may be used, including other or different confidence interval bounds of two different risk categories for defining the Slightly Increased Risk category. This includes, by way of non-limiting example, where the clinically reported cases identifying the disease are between 20-80% but where the risk-based confidence interval of 95% for these cases has a value or range between 20-100%. As another example, where the clinically reported cases identifying the disease are greater than 80% but where the risk-based confidence interval of 95% for these cases has a value or range between 20-100%. Further values and ranges that may be used for additional and/or different implementations are described herein with respect to Tables 1-7.

As a further example, in some aspects, the classification as output for the user comprises the Not Likely at Increased Risk Category. In such aspects, the outputted classification may be based upon detecting at least one of the following conditions, e.g., as further described herein for any one or more of Tables 1-7: (i) the one or more clinically relevant allelic variant genotypes as selected for the user are associated with three or more clinically reported cases, the lower percentage risk value or range is less than 20 percent defining lung or liver disease (e.g., as associated with alpha1-antitrypsin deficiency (A1ATD)) occurring in less than 20 percent of clinically reported cases of the respective one or more alleles, and a respective risk-based confidence interval between zero to 80 percent, or (ii) the one or more clinically relevant allelic variant genotypes as selected for the user are associated with three or more clinically reported cases for which lung or liver disease occurred in 20 to 80 percent of clinically reported cases of the respective one or more alleles, and a respective risk-based confidence interval between zero to 80 percent.

As a still further example, in some aspects, wherein the classification as output for the user comprises the Slightly Increased Risk Category. In such aspects, the outputted classification may be based upon detecting at least one of the following conditions, e.g., as further described herein for any one or more of Tables 1-7: (i) the one or more clinically relevant allelic variant genotypes as selected for the user are associated with three or more clinically reported cases, the middle percentage risk value or range is between 20 percent and 80 percent defining lung or liver disease (e.g., as associated with alpha1-antitrypsin deficiency (A1ATD)) occurring in 20 to 80 percent of clinically reported cases of the respective one or more alleles, and a respective risk-based confidence interval between 20 to 100 percent, or (ii) the one or more clinically relevant allelic variant genotypes as selected for the user are associated with three or more clinically reported cases, lung or liver disease occurred in at least 80 percent of clinically reported cases of the respective one or more alleles, and a respective risk-based confidence interval between 20 to 100 percent.

As a still further example, in some aspects, the classification as output for the user comprises the Increased Risk Category. In such aspects, the outputted classification may be based upon detecting at least one of the following conditions, e.g., as further described herein for any one or more of Tables 1-7: the one or more clinically relevant allelic variant genotypes as selected for the user are associated with three or more clinically reported cases, the upper percentage risk value or range is greater than 80 percent defining lung or liver disease (e.g., as associated with alpha1-antitrypsin deficiency (A1ATD)) occurring in at least 80 percent of clinically reported cases of the respective one or more alleles, and a respective risk-based confidence interval greater than 80 percent.

As a still further example, in some aspects, the classification as output for the user comprises the Unknown Risk Category. In such aspects, the outputted classification may be based upon detecting at least one of the following conditions, e.g., as further described herein for any one or more of Tables 1-7: (i) the one or more alleles of the user are one or more clinically relevant allelic variants of the PROC gene, the one or more variants of the PROC gene include one or more mutations of Arg42Cys, (ii) the one or more alleles of the user are one or more clinically relevant allelic variants of the PROC gene, the one or more variants of the PROC gene include one or more mutations of Arg51Cys, (iii) the one or more alleles of the user are one or more clinically relevant allelic variants of the PROC gene, the one or more variants of the PROC gene include one or more mutations of Val76Met, (iv) the one or more alleles of the user are one or more clinically relevant allelic variants of the PROC gene, the one or more variants of the PROC gene include one or more mutations of C 2633 G, and/or (v) the one or more alleles of the user are one or more clinically relevant allelic variants of the PROC gene, the one or more variants of the PROC gene include one or more mutations of C 2730 T.

With further reference to FIG. 2, at block 210, genetic-based biological sample analysis method 200 comprises generating, by the one or more processors (e.g., one or more processors of server 102), a user-specific genetic health risk determination for the user based on the classification of the user and the lab-based genetic analysis output.

In some aspects, the user-specific genetic health risk determination may comprise a report selected from a preset list of report types based on the one or more clinically relevant allelic variant genotypes. For example, combinations of e.g., the clinically relevant allelic variant genotypes of detected in genomic DNA of a user, the number of clinically relevant allelic variant genotypes of detected in genomic DNA of a user, etc., may result in a pre-known set of reports indicating a user-specific genetic health risk determination, as described herein, for example, with respect to Table 3 and FIG. 4.

With further reference to FIG. 2, at block 212, genetic-based biological sample analysis method 200 comprises providing, by the one or more processors (e.g., one or more processors of server 102), to the user the user-specific genetic health risk determination. For example, the user-specific genetic health risk determination can be generated and displayed by a computing device (e.g., computing device 111c) as shown and described for FIGS. 1 and 4 herein.

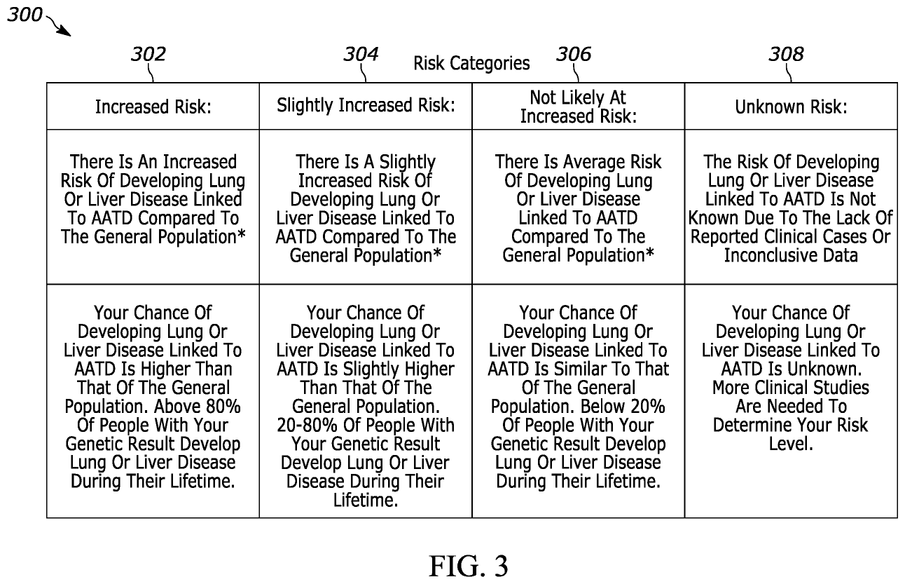

FIG. 3 illustrates an example predetermined set of risk categories 300 as may be output by a genetic risk model or otherwise classifier, in accordance with various aspects disclosed herein. As illustrated, the risk categories 300 of FIG. 3 comprise Increased Risk category 302, Slightly Increased Risk category 304, Not Likely at Increased Risk category 306, and Unknown Risk Category 308. Additionally, in some aspects the one or more alleles of the user are selected from one or more clinically relevant allelic variants of the serpin peptidase inhibitor class A member 1 (SERPINA1) gene, and the disease the genetic-based biological sample analysis system 100 is configured to detect is alpha1-antitrypsin deficiency (A1ATD) associated with lung disease and/or alpha1-antitrypsin deficiency (A1ATD) associated with liver disease.

For example, in some implementation regarding alpha1-antitrypsin deficiency (A1ATD), the genetic risk model may output Increased Risk category 302 when the user's risk of developing lung or liver disease linked to AATD is increased compared to the general population. In this example, 80% of people with the user's genetic result develop lung or liver disease during their lifetime. In some aspects, the genetic risk model may output Slightly Increased Risk category 304 when the user's risk of developing lung or liver disease linked to AATD is slightly increased compared to the general population. For example, 20-80% of people with the user's genetic result may develop lung or liver disease during their lifetime.

In some aspects, the genetic risk model may output Not Likely at Increased Risk category 306 when the user is at average risk of developing lung or liver disease linked to AATD compared to the general population. For example, the user's chance of developing lung or liver disease linked to AATD is similar to that of the general population. Furthermore, less than 20% of people with the user's genetic result may develop lung or liver disease during their lifetime. In some aspects, the genetic risk model may output Unknown Risk Category 308 when the user's risk of developing lung or liver disease linked to AATD is not known due to the lack of related clinical cases or inconclusive data. For example, a user's chance of developing lung or liver disease linked to AATD may be unknown and more clinical studies may be needed to determine a risk categorization for a specific user based on the specific user's genetic characteristics (e.g., DNA).

As described herein, the genetic risk model may output a classification (e.g., Increased Risk category 302, Slightly Increased Risk category 304, Not Likely at Increased Risk category 306, Unknown Risk Category 308) based on the lab-based genetic analysis output. In some aspects, the lab-based genetic analysis output may comprise one or more of (a) a quantity of clinically reported cases of the respective one or more alleles, (b) a percentage of clinically reported cases of the respective one or more alleles in which lung or liver disease occurred, and/or (c) a respective risk-based confidence interval.

As a specific example, Table 1 below corresponds to an implementation in which the user's risk category, as described herein, for developing lung disease (e.g., as associated with alpha1-antitrypsin deficiency (A1ATD)) depends on the number of clinically reported cases of the respective one or more alleles, the percentage of clinically reported cases of the respective one or more alleles in which lung disease occurred, and the risk-based confidence interval.

TABLE 1
General Criteria for Lung Disease Risk Categorization

| Number of | % Reported | | |
|---|---|---|---|
| reported | clinical cases | | Reported lung disease |
| clinical cases | with lung disease | 95% CI | risk category* |
| n < 3 | Any | Any | Unknown risk |
| n ≥ 3 | <20% | 0-80% | Not Likely at |
| | | | increased risk |
| | | 0-100% | Unknown risk |
| | 20-80% | 0-80% | Not Likely at |
| | | | increased risk |
| | | 20-100% | Slightly increased risk |
| | | 0-100% | Unknown risk |
| | 80% | 0-100% | Unknown risk |
| | | 20-100% | Slightly increased risk |
| | | >80% | Increased risk |

NOTE:
Exceptions to this general classification are:
All carriers with at least one PI*M allele (except PI*M/PI*Z ever-smokers) will be reported as ‘Not likely at increased risk’
PI*Z/PI*Null and PI*Null/PI*Null combinations will be assigned as ‘Increased risk’. Null variants: PI*Q0 granite falls, PI*Q0 west, PI*Q0 bellingham, PI*Q0 mattawa and PI*Q0 clayton
*The reported risk category may be updated if there is enough data to provide confidence intervals that do not overlap between two consecutive risk categories.

As another specific example, Table 2 below corresponds to an implementation in which the user's risk category, as described herein, for developing liver disease (e.g., as associated with alpha11-antitrypsin deficiency (A1ATD)) depends on the number of clinically reported cases of the respective one or more alleles, the percentage of clinically reported cases of the respective one or more alleles in which lung disease occurred, and the risk-based confidence interval.

TABLE 2
General Criteria for Liver Disease Risk Categorization

| Number of | % Reported | | |
|---|---|---|---|
| reported | clinical cases | | Reported liver disease |
| clinical cases | with liver disease | 95% CI | risk category* |
| n < 3 | Any | Any | Unknown risk |
| n ≥ 3 | <20% | 0-80% | Not Likely at |
| | | | Increased risk |
| | | 0-100% | Unknown risk |
| | 20-80% | 0-80% | Not Likely at |
| | | | Increased risk |
| | | 20-100% | Slightly increased risk |
| | | 0-100% | Unknown risk |
| | 80% | 0-100% | Unknown risk |
| | | 20-100% | Slightly increased risk |
| | | >80% | Increased risk |

NOTE:
Exceptions to this general liver disease risk classification are:
Individuals with at least one PI*Z or PI*M malton severe accumulation allele and their combinations will be reported as “Slightly Increased risk”
All the combinations including two non-accumulation alleles will be reported as “Not likely at Increased risk”. Non-accumulation alleles: PI*M, PI*M procida, PI*Q0 granite falls, PI*Q0 west, PI*QO bellingham, PI*F, PI*P lowell, PI*Q0 mattawa, PI*Q0 clayton, and PI*M heerlen
*The reported risk category may be updated if there is enough published data to provide confidence intervals that do not overlap between two consecutive risk categories

As shown in Table 1 and Table 2, in some aspects, the classification as output for the user may comprise the Increased Risk category 302 when the following is detected: one or more clinically relevant allelic variant genotypes as selected for the user are associated with three or more clinically reported cases, the upper percentage risk value or range is greater than 80 percent defining lung or liver disease (e.g., as associated with alpha1-antitrypsin deficiency (A1ATD)) occurring in at least 80 percent of clinically reported cases of the respective one or more alleles, and a respective risk-based confidence interval greater than 80 percent. Additionally, or alternatively, the classification as output for the user may comprise the Increased Risk category 302 when the following is detected: the one or more clinically relevant allelic variant genotypes as selected for the user are associated with eleven (11) or more clinically reported cases of respective other users having the one or more clinically relevant allelic variant genotypes and the disease.

As shown in Table 1 and Table 2, in some aspects, the classification as output for the user may comprise the Slightly Increased Risk category 304 when the following is detected: the one or more clinically relevant allelic variant genotypes as selected for the user are associated with three or more clinically reported cases, the middle percentage risk value or range is between 20 percent and 80 percent defining lung or liver disease (e.g., as associated with alpha1-antitrypsin deficiency (A1ATD)) occurring in 20 to 80 percent of clinically reported cases of the respective one or more alleles, and a respective risk-based confidence interval between 20 to 100 percent. Additionally, or alternatively, the classification as output for the user may comprise the Slightly Increased Risk category 304 when the following is detected: the one or more clinically relevant allelic variant genotypes as selected for the user are associated with three or more clinically reported cases, lung or liver disease occurred in at least 80 percent of clinically reported cases of the respective one or more alleles, and a respective risk-based confidence interval between 20 to 100 percent.

As shown in Table 1 and Table 2, in some aspects, the classification as output for the user may comprise the Not Likely at Increased Risk category 306 when the following is detected: the one or more clinically relevant allelic variant genotypes as selected for the user are associated with three or more clinically reported cases, the lower percentage risk value or range is less than 20 percent defining lung or liver disease (e.g., as associated with alpha1-antitrypsin deficiency (A1ATD)) occurring in less than 20 percent of clinically reported cases of the respective one or more alleles, and a respective risk-based confidence interval between zero to 80 percent. Additionally, or alternatively, the classification as output for the user may comprise the Not Likely at Increased Risk category 306 when the following is detected: the one or more clinically relevant allelic variant genotypes as selected for the user are associated with three or more clinically reported cases for which lung or liver disease occurred in 20 to 80 percent of clinically reported cases of the respective one or more alleles, and a respective risk-based confidence interval between zero to 80 percent.

As shown in Table 1 and Table 2, the classification as output for the user may comprise the Unknown Risk Category 308 when the following is detected: the one or more clinically relevant allelic variant genotypes as selected for the user are associated with two or less clinically reported cases. Additionally, or alternatively, the classification as output for the user may comprise the Unknown Risk Category 308 when the following is detected: the one or more clinically relevant allelic variant genotypes as selected for the user are associated with three or more clinically reported cases but the undetermined percentage risk value or range overlaps with at least one of the upper percentage risk value or range, the middle percentage risk value or range, or the lower percentage risk value or range.

FIG. 4 illustrates an example graphic user interface (GUI) 400 as rendered on a display screen of a user computing device depicting a user-specific genetic health risk determination of a user, in accordance with various aspects disclosed herein. For example, a user may review their genetic health risk by viewing GUI 400 on the display of the computing device 111c. GUI 400 may depict various sections indicating specific information of the user's genetic health risk.

As further illustrated in FIG. 4, GUI 400 may comprise GUI portion 402, GUI portion 404, and GUI portion 406. GUI portion 402 includes, by way of non-limiting example, output or display of user-specific genetic health risk determination, or a portion thereof, including a risk classification or category specific to the user and the identified disease e.g., “You have 2 Variants: PI*Z and PI*Z in the SERPINA1 gene linked to AATD” and may indicate to the user the determined one or more alleles selected from one or more clinically relevant allelic variant genotypes based on the genomic DNA of the user. For example, a GUI portion 402 may inform a user they are homozygous for the PI*Z variant of the SERPINA1 gene.

GUI 400 may further comprise GUI portion 404 indicating the user's risk categorization for A1ATD associated with lung disease and GUI portion 406 indicating the user's risk categorization for A1ATD associated with liver disease. For example, GUI portion 404 includes, by way of non-limiting example, output or display of user-specific genetic health risk determination, or a portion thereof, including a risk classification or category specific to the user and the identified disease, e.g., “You are at increased risk of developing lung disease linked to AATD compared to the general population,” and GUI portion 406 includes, by way of non-limiting example, output or display of user-specific genetic health risk determination, or a portion thereof, including a risk classification or category specific to the user and the identified disease, e.g., “You are at slightly increased risk of developing lung disease linked to AATD compared to the general population.”

In some implementations, the user-specific genetic health risk determination, e.g., as displayed by the computing device, may be based on a given type of report as determined from the number variants detected for a specific user, and the reported risk category. For example, the user-specific genetic health risk determination may comprise a report selected from a preset list of report types based on the one or more clinically relevant allelic variant genotypes.

As a specific example, Table 3 below corresponds to an implementation in which allele(s) of the user are selected from one or more clinically relevant allelic variant genotypes associated with an alpha1-antitrypsin deficiency (A1ATD) gene. The diseases tested for can include, by way of non-limiting example, lung disease and/or liver disease (e.g., as associated with alpha1-antitrypsin deficiency (A1ATD)).

As shown in Table 3 below, based on the number of clinically relevant allelic variants detected (0, 1, 2, or variant not determined) and the specific lung and liver disease (e.g., as associated with alpha1-antitrypsin deficiency (A1ATD)) risk categorization reported, a total of 15 types of user-specific genetic health risk determinations, or otherwise reports, can be generated.

TABLE 3

| | Number of | | | Number of clinically | |
|---|---|---|---|---|---|
| | clinically relevant | | | relevant allelic | |
| | allelic variant | | | variant genotype | |
| | genotypes detected | Reported Lung | Reported Liver | combinations | |
| Type of | in genomic DNA of | Disease Risk | Disease Risk | associated with type | Frequency of |
| Report | the user | Category | Category | of report | genetic results, % |
| 1 | 0 | Not likely at risk for | Not likely at risk for | 1 | 94.95 |
| | | AATD | AATD | | |
| 2 | 1 | Not likely at increased | Not likely at increased | 11 | 4.23 |
| | | risk | risk | | |
| 3 | 1 | Not likely at increased | Slightly increased risk | 1 | 0.002 |
| | | risk | | | |
| 4 | 1 | Not likely at increased | Unknown risk | 1 | 0.002 |
| | | risk | | | |
| 5 | 1 | Not likely at increased | Slightly increased risk | 1 | 0.780 |
| | | risk (never-smokers) | | | |
| | | Slightly increased | | | |
| | | risk (ever-smokers) | | | |
| 6 | 2 | Not likely at increased | Not likely at increased | 1 | 0.40 |
| | | risk | risk | | |
| 7 | 2 | Slightly increased risk | Not likely at increased | 1 | <0.00001 |
| | | | risk | | |
| 8 | 2 | Slightly increased risk | Slightly increased risk | 5 | 0.017 |
| 9 | 2 | Slightly increased risk | Unknown risk | 1 | <0.00001 |
| 10 | 2 | Increased risk | Not likely at increased | 15 | <0.00001 |
| | | | risk | | |
| 11 | 2 | Increased risk | Slightly increased risk | 7 | 0.0016 |
| 12 | 2 | Unknown risk | Slightly increased risk | 15 | 0.0001 |
| 13 | 2 | Unknown risk | Not likely at increased | 29 | 0.0001 |
| | | | risk | | |
| 14 | 2 | Unknown risk | Unknown risk | 31 | 0.0069 |
| 15 | Variant not determined | Not determined risk | Not determined risk | N/A | N/A |

The reported risk category shown in Table 3 above and described in FIG. 4, may correspond to the risk category output by the genetic risk model. For example, the GUI 400 of FIG. 4 corresponds to report type 11 wherein the user's risk categorization for lung disease is Increased Risk and the user's risk categorization for liver disease is Slightly Increased Risk.

In some implementations, the one or more clinically relevant allelic variant genotypes comprises one or more variants of the serpin peptidase inhibitor class A member 1 (SERPINA1) gene. For example, the generation of the lab-based genetic analysis output may comprise determining one or more alleles of the user selected from one or more clinically relevant allelic variant genotypes of the SERPINA1 gene. In some aspects, the one or more variants of the SERPINA1 gene include one or more of: (i) PI*S; (ii) PI*Z; (iii) PI*I; (iv) PI*M procida; (v) PI*M malton; (vi) PI*S iiyama; (vii) PI*Q0 granite falls; (viii) PI*Q0 west; (ix) PI*Q0 bellingham; (x) PI*F; (xi) PI*P lowell; (xii) PI*Q0 mattawa; (xiii) PI*Q0 clayton, and (xiv) PI*M Heerlen. As described herein, the user's risk categorization may be based on the one or more selected variants.

As a specific example, Table 4 below corresponds to an implementation in which allele(s) of the user are selected from one or more of the 14 clinically relevant allelic variant genotypes (variants) of the SERPINA1 gene (PI*S, PI*Z, PI*I, PI*M procida, PI*M malton, PI*S iiyama, PI*Q0 granite falls, PI*Q0 west, PI*Q0 bellingham, PI*F, PI*P lowell, PI*Q0 mattawa, PI*Q0 clayton, PI*M Heerlen) and the reported risk category for developing lung disease depend on the combination(s) (or lack thereof) of the alleles of the user.

Because a user may have no alleles (genetic result of zero), one allele (genetic result of one), or two alleles (genetic result of 2) with one or more of the 14 clinically relevant allelic variant genotypes of the SERPINA1 gene, the lab-based genetic analysis output may indicate one of 120 possible genotype combinations. Specifically, in this example, there is one genetic result with zero variants detected (wild-type homozygous), 14 genetic results with one variant detected (heterozygous), and 105 genetic results with two variants detected (homozygous or compound heterozygous). Furthermore, in some aspects, the clinically relevant allelic variant genotype may not be determined. In these aspects, the lab-based genetic analysis output may indicate one of 121 possible genotype combinations. As shown in Table 4 below, the specific genotype combination is associated with a risk categorization for developing lung disease.

TABLE 4

| | Clinically | | | | | | | | | |
|---|---|---|---|---|---|---|---|---|---|---|
| | Relevant | | | | | | | | | |
| | Allelic | | | | | Reported | % | | | |
| | Variant | | | | Reported | Clinical | Reported | | | |
| | Genotypes | | | | Clinical | Cases | Clinical | | | Reported |
| | Detected in | | Classification | | Cases | With- | Cases | | | Lung |
| | Genomic | | Group (AAT | Reported | With | Out | With | | | Disease |
| | DNA of the | Number of | plasma | Clinical | Lung | Lung | Lung | | | Risk |
| # | User | Variants | deficiency)*** | Cases | Disease | Disease | Disease | CI95% | | Category |
| 1 | PI*M/PI*M | 0 | PI*Normal/PI* | N/A | N/A | N/A | N/A | N/A | N/A | Not likely |
| | | | Normal | | | | | | | at risk |
| | | | | | | | | | | for AATD |
| 2 | PI*M/PI*I | 1 | PI*Normal/PI* | 28 | 4 | 24 | 14.30% | 6.60% | 28.30% | Not likely |
| | | | Mild | | | | | | | at increased |
| | | | | | | | | | | risk |
| 3 | PI*M/PI*M | 1 | PI*Normal/PI* | 2 | 0 | 2 | 0.00% | 0.00% | 57.50% | Not likely |
| | procida | | Severe | | | | | | | at increased |
| | | | | | | | | | | risk** |
| 4 | PI*M/PI*M | 1 | PI*Normal/PI* | 50 | 3 | 47 | 6.00% | 2.40% | 14.10% | Not likely |
| | malton | | Severe | | | | | | | at increased |
| | | | | | | | | | | risk |
| 5 | PI*M/PI*S | 1 | PI*Normal/PI* | 4 | 0 | 4 | 0.00% | 0.00% | 40.40% | Not likely |
| | iiyama | | Severe | | | | | | | at increased |
| | | | | | | | | | | risk |
| 6 | PI*M/PI*Q0 | 1 | PI*Normal/PI* | 3 | 0 | 3 | 0.00% | 0.00% | 47.40% | Not likely |
| | granite falls | | Very severe | | | | | | | at increased |
| | | | | | | | | | | risk |
| 7 | PI*M/PI*Q0 | 1 | PI*Normal/PI* | 1 | 1 | 0 | 100.00% | 27.00% | 100.00% | Not likely |
| | west | | Very severe | | | | | | | at increased |
| | | | | | | | | | | risk** |
| 8 | PI*M/PI*Q0 | 1 | PI*Normal/PI* | 3 | 0 | 3 | 0.00% | 0.00% | 47.40% | Not likely |
| | bellingham | | Very severe | | | | | | | at increased |
| | | | | | | | | | | risk |
| 9 | PI*M/PI*F | 1 | PI*Normal/PI* | 104 | 17 | 87 | 16.30% | 11.20% | 23.10% | Not likely |
| | | | Mild | | | | | | | at increased |
| | | | | | | | | | | risk |
| 10 | PI*M/PI*P | 1 | PI*Normal/PI* | 18 | 2 | 16 | 11.10% | 3.70% | 28.60% | Not likely |
| | lowell | | Severe | | | | | | | at increased |
| | | | | | | | | | | risk |
| 11 | PI*M/PI*S | 1 | PI*Normal/PI* | 792 | 101 | 691 | 12.80% | 10.90% | 14.80% | Not likely |
| | | | Mild | | | | | | | at increased |
| | | | | | | | | | | risk |
| 12 | PI*M/PI*Z | 1 | PI*Normal/PI* | 2122 | 155 | 1967 | 7.30% | 6.40% | 8.30% | Not likely |
| | | | Severe | | | | | | | at increased |
| | | | | | | | | | | risk |
| | | | | | | | | | | (never- |
| | | | | | | | | | | smokers) |
| 13 | PI*M/PI*Z | 1 | PI*Normal/PI* | 255 | 113 | 142 | 44.30% | 39.30% | 49.50% | Slightly |
| | | | Severe | | | | | | | at increased |
| | | | | | | | | | | risk |
| | | | | | | | | | | (ever- |
| | | | | | | | | | | smokers) |
| 14 | PI*M/PI*Q0 | 1 | PI*Normal/PI* | 14 | 3 | 11 | 21.40% | 8.90% | 43.20% | Not likely |
| | mattawa | | Very severe | | | | | | | at increased |
| | | | | | | | | | | risk |
| 15 | PI*M/PI*Q0 | 1 | PI*Normal/PI* | 1 | 0 | 1 | 0.00% | 0.00% | 73.00% | Not likely |
| | clayton | | Very severe | | | | | | | at increased |
| | | | | | | | | | | risk** |
| 16 | PI*M/PI*M | 1 | PI*Normal/PI* | 11 | 3 | 8 | 27.30% | 11.50% | 52.10% | Not likely |
| | heerlen | | Severe | | | | | | | at increased |
| | | | | | | | | | | risk |
| 17 | PI*Z/PI*I | 2 | PI*Mild/PI*Severe | 4 | 4 | 0 | 100.00% | 59.60% | 100.00% | Slightly |
| | | | | | | | | | | at increased |
| | | | | | | | | | | risk |
| 18 | PI*Z/PI*M | 2 | PI*Severe/PI* | 2 | 2 | 0 | 100.00% | 42.50% | 100.00% | Unknown |
| | procida | | Severe | | | | | | | risk |
| 19 | PI*Z/PI*M | 2 | PI*Severe/PI* | 13 | 13 | 0 | 100.00% | 82.80% | 100.00% | Increased |
| | malton | | Severe | | | | | | | risk |
| 20 | PI*Z/PI*S | 2 | PI*Severe/PI* | 0 | 0 | 0 | N/A | N/A | N/A | Unknown |
| | iiyama | | Severe | | | | | | | risk |
| 21 | PI*Z/PI*Q0 | 2 | PI*Severe/PI* | 3 | 3 | 0 | 100.00% | 52.60% | 100.00% | Increased |
| | granite falls | | Very severe | | | | | | | risk** |
| 22 | PI*Z/PI*Q0 | 2 | PI*Severe/PI* | 0 | 0 | 0 | N/A | N/A | N/A | Increased |
| | west | | Very severe | | | | | | | risk** |
| 23 | PI*Z/PI*Q0 | 2 | PI*Severe/PI* | 1 | 1 | 0 | 100.00% | 27.00% | 100.00% | Increased |
| | bellingham | | Very severe | | | | | | | risk** |
| 24 | PI*F/PI*Z | 2 | PI*Mild/PI*Severe | 15 | 13 | 2 | 86.70% | 66.60% | 95.50% | Slightly |
| | | | | | | | | | | at increased |
| | | | | | | | | | | risk |
| 25 | PI*Z/PI*P | 2 | PI*Severe/PI* | 7 | 7 | 0 | 100.00% | 72.10% | 100.00% | Slightly |
| | lowell | | Severe | | | | | | | at increased |
| | | | | | | | | | | risk |
| 26 | PI*S/PI*Z | 2 | PI*Mild/PI* | 497 | 269 | 228 | 54.10% | 50.40% | 57.80% | Slightly |
| | | | Severe | | | | | | | at increased |
| | | | | | | | | | | risk |
| 27 | PI*Z/PI*Z | 2 | PI*Severe/PI* | 3405 | 2775 | 630 | 81.50% | 80.40% | 82.60% | Increased |
| | | | Severe | | | | | | | risk |
| 28 | PI*Z/PI*Q0 | 2 | PI*Severe/PI* | 1 | 1 | 0 | 100.00% | 27.00% | 100.00% | Increased |
| | mattawa | | Very severe | | | | | | | risk** |
| 29 | PI*Z/PI*Q0 | 2 | PI*Severe/PI* | 0 | 0 | 0 | N/A | N/A | N/A | Increased |
| | clayton | | Very severe | | | | | | | risk** |
| 30 | PI*Z/PI*M | 2 | PI*Severe/PI* | 2 | 2 | 0 | 100.00% | 42.50% | 100.00% | Unknown |
| | heerlen | | Severe | | | | | | | risk |
| 31 | PI*S/PI*I | 2 | PI*Mild/PI*Mild | 2 | 0 | 2 | 0.00% | 0.00% | 57.50% | Unknown |
| | | | | | | | | | | risk |
| 32 | PI*S/PI*M | 2 | PI*Mild/PI* | 0 | 0 | 0 | N/A | N/A | N/A | Unknown |
| | procida | | Severe | | | | | | | risk |
| 33 | PI*S/PI*M | 2 | PI*Mild/PI* | 1 | 1 | 0 | 100.00% | 27.00% | 100.00% | Unknown |
| | malton | | Severe | | | | | | | risk |
| 34 | PI*S/PI*S | 2 | PI*Mild/PI* | 0 | 0 | 0 | N/A | N/A | N/A | Unknown |
| | iiyama | | Severe | | | | | | | risk |
| 35 | PI*S/PI*Q0 | 2 | PI*Mild/PI* | 0 | 0 | 0 | N/A | N/A | N/A | Unknown |
| | granite falls | | Very severe | | | | | | | risk |
| 36 | PI*S/PI*Q0 | 2 | PI*Mild/PI* | 0 | 0 | 0 | N/A | N/A | N/A | Unknown |
| | west | | Very severe | | | | | | | risk |
| 37 | PI*S/PI*Q0 | 2 | PI*Mild/PI* | 0 | 0 | 0 | N/A | N/A | N/A | Unknown |
| | bellingham | | Very severe | | | | | | | risk |
| 38 | PI*F/PI*S | 2 | PI*Mild/PI*Mild | 1 | 0 | 1 | 0.00% | 0.00% | 73.00% | Unknown |
| | | | | | | | | | | risk |
| 39 | PI*S/PI*P | 2 | PI*Mild/PI* | 2 | 1 | 1 | 50.00% | 12.10% | 87.90% | Unknown |
| | lowell | | Severe | | | | | | | risk |
| 40 | PI*S/PI*S | 2 | PI*Mild/PI*Mild | 46 | 4 | 42 | 8.70% | 4.00% | 18.00% | Not likely |
| | | | | | | | | | | at increased |
| | | | | | | | | | | risk |
| 41 | PI*S/PI*Q0 | 2 | PI*Mild/PI* | 0 | 0 | 0 | N/A | N/A | N/A | Unknown |
| | mattawa | | Very severe | | | | | | | risk |
| 42 | PI*S/PI*Q0 | 2 | PI*Mild/PI* | 2 | 1 | 1 | 50.00% | 12.10% | 87.90% | Unknown |
| | clayton | | Very severe | | | | | | | risk |
| 43 | PI*S/PI*M | 2 | PI*Mild/PI* | 2 | 1 | 1 | 50.00% | 12.10% | 87.90% | Unknown |
| | heerlen | | Severe | | | | | | | risk |
| 44 | PI*I/PI*I | 2 | PI*Mild/PI*Mild | 0 | 0 | 0 | N/A | N/A | N/A | Unknown |
| | | | | | | | | | | risk |
| 45 | PI*I/PI*M | 2 | PI*Mild/PI* | 0 | 0 | 0 | N/A | N/A | N/A | Unknown |
| | procida | | Severe | | | | | | | risk |
| 46 | PI*I/PI*M | 2 | PI*Mild/PI* | 0 | 0 | 0 | N/A | N/A | N/A | Unknown |
| | malton | | Severe | | | | | | | risk |
| 47 | PI*I/PI*S | 2 | PI*Mild/PI* | 0 | 0 | 0 | N/A | N/A | N/A | Unknown |
| | iiyama | | Severe | | | | | | | risk |
| 48 | PI*I/PI*Q0 | 2 | PI*Mild/PI* | 0 | 0 | 0 | N/A | N/A | N/A | Unknown |
| | granite falls | | Very severe | | | | | | | risk |
| 49 | PI*I/PI*Q0 | 2 | PI*Mild/PI* | 0 | 0 | 0 | N/A | N/A | N/A | Unknown |
| | west | | Very severe | | | | | | | risk |
| 50 | PI*I/PI*Q0 | 2 | PI*Mild/PI* | 0 | 0 | 0 | N/A | N/A | N/A | Unknown |
| | bellingham | | Very severe | | | | | | | risk |
| 51 | PI*F/PI*I | 2 | PI*Mild/PI*Mild | 0 | 0 | 0 | N/A | N/A | N/A | Unknown |
| | | | | | | | | | | risk |
| 52 | PI*I/PI*P | 2 | PI*Mild/PI* | 0 | 0 | 0 | N/A | N/A | N/A | Unknown |
| | lowell | | Severe | | | | | | | risk |
| 53 | PI*I/PI*Q0 | 2 | PI*Mild/PI* | 0 | 0 | 0 | N/A | N/A | N/A | Unknown |
| | mattawa | | Very severe | | | | | | | risk |
| 54 | PI*I/PI*Q0 | 2 | PI*Mild/PI* | 0 | 0 | 0 | N/A | N/A | N/A | Unknown |
| | clayton | | Very severe | | | | | | | risk |
| 55 | PI*I/PI*M | 2 | PI*Mild/PI* | 0 | 0 | 0 | N/A | N/A | N/A | Unknown |
| | heerlen | | Severe | | | | | | | risk |
| 56 | PI*F/PI*M | 2 | PI*Mild/PI* | 0 | 0 | 0 | N/A | N/A | N/A | Unknown |
| | procida | | Severe | | | | | | | risk |
| 57 | PI*F/PI*M | 2 | PI*Mild/PI*Severe | 0 | 0 | 0 | N/A | N/A | N/A | Unknown |
| | malton | | | | | | | | | risk |
| 58 | PI*F/PI*S | 2 | PI*Mild/PI*Severe | 0 | 0 | 0 | N/A | N/A | N/A | Unknown |
| | iiyama | | | | | | | | | risk |
| 59 | PI*F/PI*Q0 | 2 | PI*Mild/PI*Very | 0 | 0 | 0 | N/A | N/A | N/A | Unknown |
| | granite falls | | severe | | | | | | | risk |
| 60 | PI*F/PI*Q0 | 2 | PI*Mild/PI*Very | 0 | 0 | 0 | N/A | N/A | N/A | Unknown |
| | west | | severe | | | | | | | risk |
| 61 | PI*F/PI*Q0 | 2 | PI*Mild/PI*Very | 0 | 0 | 0 | N/A | N/A | N/A | Unknown |
| | bellingham | | severe | | | | | | | risk |
| 62 | PI*F/PI*F | 2 | PI*Mild/PI*Mild | 1 | 1 | 0 | 100.00% | 27.00% | 100.00% | Unknown |
| | | | | | | | | | | risk |
| 63 | PI*F/PI*P | 2 | PI*Mild/PI*Very | 0 | 0 | 0 | N/A | N/A | N/A | Unknown |
| | lowell | | severe | | | | | | | risk |
| 64 | PI*F/PI*Q0 | 2 | PI*Mild/PI*Very | 0 | 0 | 0 | N/A | N/A | N/A | Unknown |
| | mattawa | | severe | | | | | | | risk |
| 65 | PI*F/PI*Q0 | 2 | PI*Mild/PI*Very | 1 | 1 | 0 | 100.00% | 27.00% | 100.00% | Unknown |
| | clayton | | severe | | | | | | | risk |
| 66 | PI*F/PI*M | 2 | PI*Mild/PI*Severe | 0 | 0 | 0 | N/A | N/A | N/A | Unknown |
| | heerlen | | | | | | | | | risk |
| 67 | PI*M | 2 | PI*Severe/PI* | 2 | 2 | 0 | 100.00% | 42.50% | 100.00% | Unknown |
| | procida/PI* | | Severe | | | | | | | risk |
| | M procida | | | | | | | | | |
| 68 | PI*M | 2 | PI*Severe/PI* | 0 | 0 | 0 | N/A | N/A | N/A | Unknown |
| | malton/PI* | | Severe | | | | | | | risk |
| | M procida | | | | | | | | | |
| 69 | PI*M | 2 | PI*Severe/PI* | 0 | 0 | 0 | N/A | N/A | N/A | Unknown |
| | procida/PI* | | Severe | | | | | | | risk |
| | S iiyama | | | | | | | | | |
| 70 | PI*M | 2 | PI*Severe/PI* | 0 | 0 | 0 | N/A | N/A | N/A | Unknown |
| | procida/PI* | | Very severe | | | | | | | risk |
| | Q0 granite | | | | | | | | | |
| | falls | | | | | | | | | |
| 71 | PI*M | 2 | PI*Severe/PI* | 0 | 0 | 0 | N/A | N/A | N/A | Unknown |
| | procida/PI* | | Very severe | | | | | | | risk |
| | Q0 west | | | | | | | | | |
| 72 | PI*M | 2 | PI*Severe/PI* | 0 | 0 | 0 | N/A | N/A | N/A | Unknown |
| | procida/PI* | | Very severe | | | | | | | risk |
| | Q0 bellingham | | | | | | | | | |
| 73 | PI*M | 2 | PI*Severe/PI* | 0 | 0 | 0 | N/A | N/A | N/A | Unknown |
| | procida/PI* | | Severe | | | | | | | risk |
| | P lowell | | | | | | | | | |
| 74 | PI*M | 2 | PI*Severe/PI* | 0 | 0 | 0 | N/A | N/A | N/A | Unknown |
| | procida/PI* | | Very severe | | | | | | | risk |
| | Q0 mattawa | | | | | | | | | |
| 75 | PI*M | 2 | PI*Severe/PI* | 0 | 0 | 0 | N/A | N/A | N/A | Unknown |
| | procida/PI* | | Very severe | | | | | | | risk |
| | Q0 clayton | | | | | | | | | |
| 76 | PI*M | 2 | PI*Severe/PI* | 0 | 0 | 0 | N/A | N/A | N/A | Unknown |
| | procida/PI* | | Severe | | | | | | | risk |
| | M heerlen | | | | | | | | | |
| 77 | PI*M | 2 | PI*Severe/PI* | 24 | 19 | 5 | 79.20% | 63.00% | 89.50% | Slightly |
| | malton/PI* | | Severe | | | | | | | at increased |
| | M malton | | | | | | | | | risk |
| 78 | PI*M | 2 | PI*Severe/PI* | 0 | 0 | 0 | N/A | N/A | N/A | Unknown |
| | malton/PI*S | | Severe | | | | | | | risk |
| | iiyama | | | | | | | | | |
| 79 | PI*M | 2 | PI*Severe/PI* | 0 | 0 | 0 | N/A | N/A | N/A | Unknown |
| | malton/PI*Q0 | | Very severe | | | | | | | risk |
| | granite | | | | | | | | | |
| | falls | | | | | | | | | |
| 80 | PI*M | 2 | PI*Severe/PI* | 0 | 0 | 0 | N/A | N/A | N/A | Unknown |
| | malton/PI*Q0 | | Very severe | | | | | | | risk |
| | west | | | | | | | | | |
| 81 | PI*M | 2 | PI*Severe/PI* | 0 | 0 | 0 | N/A | N/A | N/A | Unknown |
| | malton/PI*Q0 | | Very severe | | | | | | | risk |
| | bellingham | | | | | | | | | |
| 82 | PI*M | 2 | PI*Severe/PI* | 0 | 0 | 0 | N/A | N/A | N/A | Unknown |
| | malton/PI*P | | Severe | | | | | | | risk |
| | lowell | | | | | | | | | |
| 83 | PI*M | 2 | PI*Severe/PI* | 2 | 1 | 1 | 50.00% | 12.10% | 87.90% | Unknown |
| | malton/PI*Q0 | | Very severe | | | | | | | risk |
| | mattawa | | | | | | | | | |
| 84 | PI*M | 2 | PI*Severe/PI* | 1 | 1 | 0 | 100.00% | 27.00% | 100.00% | Unknown |
| | malton/PI*Q0 | | Very severe | | | | | | | risk |
| | clayton | | | | | | | | | |
| 85 | PI*M | 2 | PI*Severe/PI* | 0 | 0 | 0 | N/A | N/A | N/A | Unknown |
| | malton/PI* | | Severe | | | | | | | risk |
| | M heerlen | | | | | | | | | |
| 86 | PI*S | 2 | PI*Severe/PI* | 3 | 3 | 0 | 100.00% | 52.60% | 100.00% | Slightly |
| | iiyama/PI*S | | Severe | | | | | | | at increased |
| | iiyama | | | | | | | | | risk |
| 87 | PI*Q0 | 2 | PI*Severe/PI* | 0 | 0 | 0 | N/A | N/A | N/A | Unknown |
| | granite | | Very severe | | | | | | | risk |
| | falls/PI*S | | | | | | | | | |
| | iiyama | | | | | | | | | |
| 88 | PI*S | 2 | PI*Severe/PI* | 0 | 0 | 0 | N/A | N/A | N/A | Unknown |
| | iiyama/PI*Q0 | | Very severe | | | | | | | risk |
| | west | | | | | | | | | |
| 89 | PI*S | 2 | PI*Severe/PI* | 0 | 0 | 0 | N/A | N/A | N/A | Unknown |
| | iiyama/PI*Q0 | | Very severe | | | | | | | risk |
| | bellingham | | | | | | | | | |
| 90 | PI*P | 2 | PI*Severe/PI* | 0 | 0 | 0 | N/A | N/A | N/A | Unknown |
| | lowell/PI*S | | Severe | | | | | | | risk |
| | iiyama | | | | | | | | | |
| 91 | PI*S | 2 | PI*Severe/PI* | 0 | 0 | 0 | N/A | N/A | N/A | Unknown |
| | iiyama/PI*Q0 | | Very severe | | | | | | | risk |
| | mattawa | | | | | | | | | |
| 92 | PI*S | 2 | PI*Severe/PI* | 2 | 2 | 0 | 100.00% | 42.50% | 100.00% | Unknown |
| | iiyama/PI*Q0 | | Very severe | | | | | | | risk |
| | clayton | | | | | | | | | |
| 93 | PI*M | 2 | PI*Severe/PI* | 0 | 0 | 0 | N/A | N/A | N/A | Unknown |
| | heerlen/PI*S | | Severe | | | | | | | risk |
| | iiyama | | | | | | | | | |
| 94 | PI*Q0 | 2 | PI*Very | 4 | 4 | 0 | 100.00% | 59.60% | 100.00% | Increased |
| | granite | | severe/PI*Very | | | | | | | risk** |
| | falls/PI*Q0 | | severe | | | | | | | |
| | granite falls | | | | | | | | | |
| 95 | PI*Q0 | 2 | PI*Very | 0 | 0 | 0 | N/A | N/A | N/A | Increased |
| | granite | | severe/PI*Very | | | | | | | risk** |
| | falls/PI*Q0 | | severe | | | | | | | |
| | west | | | | | | | | | |
| 96 | PI*Q0 | 2 | PI*Very | 0 | 0 | 0 | N/A | N/A | N/A | Increased |
| | bellingham/ | | severe/PI*Very | | | | | | | risk** |
| | PI*Q0 | | severe | | | | | | | |
| | granite falls | | | | | | | | | |
| 97 | PI*P | 2 | PI*Severe/PI* | 0 | 0 | 0 | N/A | N/A | N/A | Unknown |
| | lowell/PI*Q0 | | Very severe | | | | | | | risk |
| | granite | | | | | | | | | |
| | falls | | | | | | | | | |
| 98 | PI*Q0 | 2 | PI*Very | 0 | 0 | 0 | N/A | N/A | N/A | Increased |
| | granite | | severe/PI*Very | | | | | | | risk** |
| | falls/PI*Q0 | | severe | | | | | | | |
| | mattawa | | | | | | | | | |
| 99 | PI*Q0 | 2 | PI*Very | 0 | 0 | 0 | N/A | N/A | N/A | Increased |
| | clayton/PI* | | severe/PI*Very | | | | | | | risk** |
| | Q0 granite | | severe | | | | | | | |
| | falls | | | | | | | | | |
| 100 | PI*M | 2 | PI*Severe/PI* | 1 | 1 | 0 | 100.00% | 27.00% | 100.00% | Unknown |
| | heerlen/PI* | | Very severe | | | | | | | risk |
| | Q0 granite | | | | | | | | | |
| | falls | | | | | | | | | |
| 101 | PI*Q0 | 2 | PI*Very | 0 | 0 | 0 | N/A | N/A | N/A | Increased |
| | west/PI*Q0 | | severe/PI*Very | | | | | | | risk** |
| | west | | severe | | | | | | | |
| 102 | PI*Q0 | 2 | PI*Very | 0 | 0 | 0 | N/A | N/A | N/A | Increased |
| | bellingham/ | | severe/PI*Very | | | | | | | risk** |
| | PI*Q0 west | | severe | | | | | | | |
| 103 | PI*P | 2 | PI*Severe/PI* | 0 | 0 | 0 | N/A | N/A | N/A | Unknown |
| | lowell/PI*Q0 | | Very severe | | | | | | | risk |
| | west | | | | | | | | | |
| 104 | PI*Q0 | 2 | PI*Very | 0 | 0 | 0 | N/A | N/A | N/A | Increased |
| | mattawa/PI* | | severe/PI*Very | | | | | | | risk** |
| | Q0 west | | severe | | | | | | | |
| 105 | PI*Q0 | 2 | PI*Very | 0 | 0 | 0 | N/A | N/A | N/A | Increased |
| | clayton/PI* | | severe/PI*Very | | | | | | | risk** |
| | Q0 west | | severe | | | | | | | |
| 106 | PI*M | 2 | PI*Severe/PI* | 0 | 0 | 0 | N/A | N/A | N/A | Unknown |
| | heerlen/PI* | | Very severe | | | | | | | risk |
| | Q0 west | | | | | | | | | |
| 107 | PI*Q0 | 2 | PI*Very | 6 | 6 | 0 | 100.00% | 68.90% | 100.00% | Increased |
| | bellingham/ | | severe/PI*Very | | | | | | | risk** |
| | PI*Q0 | | severe | | | | | | | |
| | bellingham | | | | | | | | | |
| 108 | PI*P | 2 | PI*Severe/PI* | 1 | 0 | 1 | 0.00% | 0.00% | 73.00% | Unknown |
| | lowell/PI*Q0 | | Very severe | | | | | | | risk |
| | bellingham | | | | | | | | | |
| 109 | PI*Q0 | 2 | PI*Very | 2 | 2 | 0 | 100.00% | 42.50% | 100.00% | Increased |
| | bellingham/ | | severe/PI*Very | | | | | | | risk** |
| | PI*Q0 | | severe | | | | | | | |
| | mattawa | | | | | | | | | |
| 110 | PI*Q0 | 2 | PI*Very | 0 | 0 | 0 | N/A | N/A | N/A | Increased |
| | bellingham/ | | severe/PI*Very | | | | | | | risk** |
| | PI*Q0 | | severe | | | | | | | |
| | clayton | | | | | | | | | |
| 111 | PI*M | 2 | PI*Severe/PI* | 0 | 0 | 0 | N/A | N/A | N/A | Unknown |
| | heerlen/PI* | | Very severe | | | | | | | risk |
| | Q0 | | | | | | | | | |
| | bellingham | | | | | | | | | |
| 112 | PI*P | 2 | PI*Severe/PI* | 1 | 1 | 0 | 100.00% | 27.00% | 100.00% | Unknown |
| | lowell/PI*P | | Severe | | | | | | | risk |
| | lowell | | | | | | | | | |
| 113 | PI*P | 2 | PI*Severe/PI* | 0 | 0 | 0 | N/A | N/A | N/A | Unknown |
| | lowell/PI*Q0 | | Very severe | | | | | | | risk |
| | mattawa | | | | | | | | | |
| 114 | PI*P | 2 | PI*Severe/PI* | 0 | 0 | 0 | N/A | N/A | N/A | Unknown |
| | lowell/PI*Q0 | | Very severe | | | | | | | risk |
| | clayton | | | | | | | | | |
| 115 | PI*P | 2 | PI*Severe/PI* | 0 | 0 | 0 | N/A | N/A | N/A | Unknown |
| | lowell/PI*M | | Severe | | | | | | | risk |
| | heerlen | | | | | | | | | |
| 116 | PI*Q0 | 2 | PI*Very | 3 | 3 | 0 | 100.00% | 52.60% | 100.00% | Increased |
| | mattawa/PI* | | severe/PI*Very | | | | | | | risk** |
| | Q0 mattawa | | severe | | | | | | | |
| 117 | PI*Q0 | 2 | PI*Very | 0 | 0 | 0 | N/A | N/A | N/A | Increased |
| | clayton/PI* | | severe/PI*Very | | | | | | | risk** |
| | Q0 mattawa | | severe | | | | | | | |
| 118 | PI*M | 2 | PI*Severe/PI* | 0 | 0 | 0 | N/A | N/A | N/A | Unknown |
| | heerlen/PI* | | Very severe | | | | | | | risk |
| | Q0 mattawa | | | | | | | | | |
| 119 | PI*Q0 | 2 | PI*Very | 0 | 0 | 0 | N/A | N/A | N/A | Increased |
| | clayton/PI* | | severe/PI*Very | | | | | | | risk** |
| | Q0 clayton | | severe | | | | | | | |
| 120 | PI*M | 2 | PI*Severe/PI* | 1 | 1 | 0 | 100.00% | 27.00% | 100.00% | Unknown |
| | heerlen/PI* | | Very severe | | | | | | | risk |
| | Q0 clayton | | | | | | | | | |
| 121 | PI*M | 2 | PI*Severe/PI* | 5 | 5 | 0 | 100.00% | 64.90% | 100.00% | Slightly |
| | heerlen/PI* | | Severe | | | | | | | at increased |
| | M heerlen | | | | | | | | | risk |

***NOTE:
Normal: PI*M (80-120%); Mild variants: PI*S, PI*I, PI*F (35-79%); Severe variants: PI*P lowell, PI*Z, PI*M malton, PI*M procida, PI*S iiyama, PI*M herleen (1-34%); Very Severe variants (PI*Null) (PI*Q0): PI*Q0 granite falls, PI*Q0 west, PI*Q0 bellingham, PI*Q0 mattawa, PI*Q0 clayton (no active AAT, or less than 1%).
**NOTE:
Exceptions to the general classification: All carriers (except PI*M/PI*Z ever-smokers) are reported as “Not likely at increased risk” PI*Z/PI*Null and PI*Null/PI*Null combinations are assigned as “Increased risk”. Null variants: PI*Q0 granite falls, PI*Q0 west, PI*Q0 bellingham, PI*Q0 mattawa and PI*Q0 clayton.

As another specific example, Table 5 below corresponds to an implementation in which allele(s) of the user are selected from one or more of the 14 clinically relevant allelic variant genotypes (variants) of the SERPINA1 gene (Pc*, PI*, PI*I, PI*M procida, PI*M malton, PI*S iiyama, PI*Q0 granite falls, PI*Q0 west, PI*Q0 bellingham, PI*F, PI*P lowell, PI*Q0 mattawa, PI*Q0 clayton, PI*M Heerlen) and the reported risk category for developing liver disease depend on the combination(s) (or lack thereof) of the alleles of the user.

TABLE 5

| | Clinically | | | | | | | | | |
|---|---|---|---|---|---|---|---|---|---|---|
| | Relevant | | | | | | | | | |
| | Allelic | | | | | Reported | % | | | |
| | Variant | | | | Reported | Clinical | Reported | | | |
| | Genotypes | | | | Clinical | Cases | Clinical | | | Reported |
| | Detected in | | Classification | | Cases | With- | Cases | | | Liver |
| | Genomic | | Group (AAT | Reported | With | Out | With | | | Disease |
| | DNA of the | Number of | liver | Clinical | Liver | Liver | Liver | | | Risk |
| # | User | Variants | accumulation)*** | Cases | Disease | Disease | Disease | CI95% | | Category |
| 1 | PI*M/PI*M | 0 | PI*Without | N/A | N/A | N/A | N/A | N/A | N/A | Not likely |
| | | | accumulation | | | | | | | at risk |
| | | | (PI*M)/PI*Without | | | | | | | for AATD |
| | | | accumulation | | | | | | | |
| | | | (PI*M) | | | | | | | |
| 2 | PI*M/PI*I | 1 | PI*Without | 24 | 0 | 24 | 0.00% | 0.00% | 10.10% | Not likely |
| | | | accumulation | | | | | | | at increased |
| | | | (PI*M)/PI*Mild | | | | | | | risk |
| | | | accumulation | | | | | | | |
| 3 | PI*M/PI*M | 1 | PI*Without | 4 | 1 | 3 | 25.00% | 5.80% | 64.40% | Not likely |
| | procida | | accumulation | | | | | | | at increased |
| | | | (PI*M)/ | | | | | | | risk |
| | | | PI*Without | | | | | | | |
| | | | accumulation | | | | | | | |
| 4 | PI*M/PI*M | 1 | PI*Without | 26 | 12 | 14 | 46.2% | 31.2% | 61.8% | Slightly |
| | malton | | accumulation | | | | | | | at increased |
| | | | (PI*M)/ | | | | | | | risk |
| | | | PI*Severe | | | | | | | |
| | | | accumulation | | | | | | | |
| 5 | PI*M/PI*S | 1 | PI*Without | 0 | 0 | 0 | N/A | N/A | N/A | Unknown |
| | iiyama | | accumulation | | | | | | | risk |
| | | | (PI*M)/ | | | | | | | |
| | | | PI*Severe | | | | | | | |
| | | | accumulation | | | | | | | |
| 6 | PI*M/PI*Q0 | 1 | PI*Without | 0 | 0 | 0 | N/A | N/A | N/A | Not likely |
| | granite falls | | accumulation | | | | | | | at increased |
| | | | (PI*M)/ | | | | | | | risk** |
| | | | PI*Without | | | | | | | |
| | | | accumulation | | | | | | | |
| 7 | PI*M/PI*Q0 | 1 | PI*Without | 0 | 0 | 0 | N/A | N/A | N/A | Not likely |
| | west | | accumulation | | | | | | | at increased |
| | | | (PI*M)/ | | | | | | | risk** |
| | | | PI*Without | | | | | | | |
| | | | accumulation | | | | | | | |
| 8 | PI*M/PI*Q0 | 1 | PI*Without | 0 | 0 | 0 | N/A | N/A | N/A | Not likely |
| | bellingham | | accumulation | | | | | | | at increased |
| | | | (PI*M)/ | | | | | | | risk** |
| | | | PI*Without | | | | | | | |
| | | | accumulation | | | | | | | |
| 9 | PI*M/PI*F | 1 | PI*Without | 78 | 0 | 78 | 0.00% | 0.00% | 3.40% | Not likely |
| | | | accumulation | | | | | | | at increased |
| | | | (PI*M)/ | | | | | | | risk |
| | | | PI*Without | | | | | | | |
| | | | accumulation | | | | | | | |
| 10 | PI*M/PI*P | 1 | PI*Without | 1 | 0 | 1 | 0.00% | 0.00% | 73.00% | Not likely |
| | lowell | | accumulation | | | | | | | at increased |
| | | | (PI*M)/ | | | | | | | risk** |
| | | | PI*Without | | | | | | | |
| | | | accumulation | | | | | | | |
| 11 | PI*M/PI*S | 1 | PI*Without | 1246 | 122 | 1124 | 9.80% | 8.50% | 11.30% | Not likely |
| | | | accumulation | | | | | | | at increased |
| | | | (PI*M)/PI*Mild | | | | | | | risk |
| | | | accumulation | | | | | | | |
| 12 | PI*M/PI*Z | 1 | PI*Without | 1379 | 431 | 948 | 31.30% | 29.20% | 33.30% | Slightly |
| | | | accumulation | | | | | | | at increased |
| | | | (PI*M)/ | | | | | | | risk |
| | | | PI*Severe | | | | | | | |
| | | | accumulation | | | | | | | |
| 13 | PI*M/PI*Z | 1 | PI*Without | 1379 | 431 | 948 | 31.30% | 29.20% | 33.30% | Slightly |
| | | | accumulation | | | | | | | at increased |
| | | | (PI*M)/ | | | | | | | risk |
| | | | PI*Severe | | | | | | | |
| | | | accumulation | | | | | | | |
| 14 | PI*M/PI*Q0 | 1 | PI*Without | 14 | 0 | 14 | 0.00% | 0.00% | 16.20% | Not likely |
| | mattawa | | accumulation | | | | | | | at increased |
| | | | (PI*M)/ | | | | | | | risk |
| | | | PI*Without | | | | | | | |
| | | | accumulation | | | | | | | |
| 15 | PI*M/PI*Q0 | 1 | PI*Without | 0 | 0 | 0 | N/A | N/A | N/A | Not likely |
| | clayton | | accumulation | | | | | | | at increased |
| | | | (PI*M)/ | | | | | | | risk** |
| | | | PI*Without | | | | | | | |
| | | | accumulation | | | | | | | |
| 16 | PI*M/PI*M | 1 | PI*Without | 2 | 1 | 1 | 50.00% | 12.10% | 87.90% | Not likely |
| | heerlen | | accumulation | | | | | | | at increased |
| | | | (PI*M)/ | | | | | | | risk** |
| | | | PI*Without | | | | | | | |
| | | | accumulation | | | | | | | |
| 17 | PI*Z/PI*I | 2 | PI*Mild | 4 | 2 | 2 | 50.00% | 18.20% | 81.80% | Slightly |
| | | | accumulation/PI* | | | | | | | at increased |
| | | | Severe | | | | | | | risk** |
| | | | accumulation | | | | | | | |
| 18 | PI*Z/PI*M | 2 | PI*Without | 2 | 1 | 1 | 50.00% | 12.10% | 87.90% | Slightly |
| | procida | | accumulation/PI* | | | | | | | at increased |
| | | | Severe | | | | | | | risk** |
| | | | accumulation | | | | | | | |
| 19 | PI*Z/PI*M | 2 | PI*Severe | 10 | 1 | 9 | 10.00% | 2.30% | 34.80% | Slightly |
| | malton | | accumulation/PI* | | | | | | | at increased |
| | | | Severe | | | | | | | risk** |
| | | | accumulation | | | | | | | |
| 20 | PI*Z/PI*S | 2 | PI*Severe | 0 | 0 | 0 | N/A | N/A | N/A | Slightly |
| | iiyama | | accumulation/PI* | | | | | | | at increased |
| | | | Severe | | | | | | | risk** |
| | | | accumulation | | | | | | | |
| 21 | PI*Z/PI*Q0 | 2 | PI*Without | 0 | 0 | 0 | N/A | N/A | N/A | Slightly |
| | granite falls | | accumulation/PI* | | | | | | | at increased |
| | | | Severe | | | | | | | risk** |
| | | | accumulation | | | | | | | |
| 22 | PI*Z/PI*Q0 | 2 | PI*Without | 0 | 0 | 0 | N/A | N/A | N/A | Slightly |
| | west | | accumulation/PI* | | | | | | | at increased |
| | | | Severe | | | | | | | risk** |
| | | | accumulation | | | | | | | |
| 23 | PI*Z/PI*Q0 | 2 | PI*Without | 1 | 0 | 1 | 0.00% | 0.00% | 73.00% | Slightly |
| | bellingham | | accumulation/PI* | | | | | | | at increased |
| | | | Severe | | | | | | | risk** |
| | | | accumulation | | | | | | | |
| 24 | PI*F/PI*Z | 2 | PI*Without | 8 | 2 | 6 | 25.00% | 8.70% | 54.00% | Slightly |
| | | | accumulation/PI* | | | | | | | at increased |
| | | | Severe | | | | | | | risk** |
| | | | accumulation | | | | | | | |
| 25 | PI*Z/PI*P | 2 | PI*Without | 5 | 2 | 3 | 40.00% | 14.30% | 72.80% | Slightly |
| | lowell | | accumulation/PI* | | | | | | | at increased |
| | | | Severe | | | | | | | risk** |
| | | | accumulation | | | | | | | |
| 26 | PI*S/PI*Z | 2 | PI*Mild | 498 | 139 | 359 | 27.90% | 24.70% | 31.30% | Slightly |
| | | | accumulation/PI* | | | | | | | at increased |
| | | | Severe | | | | | | | risk** |
| | | | accumulation | | | | | | | |
| 27 | PI*Z/PI*Z | 2 | PI*Severe | 4437 | 1199 | 3238 | 27.00% | 25.90% | 28.10% | Slightly |
| | | | accumulation/PI* | | | | | | | at increased |
| | | | Severe | | | | | | | risk |
| | | | accumulation | | | | | | | |
| 28 | PI*Z/PI*Q0 | 2 | PI*Without | 0 | 0 | 0 | N/A | N/A | N/A | Slightly |
| | mattawa | | accumulation/PI* | | | | | | | at increased |
| | | | Severe | | | | | | | risk** |
| | | | accumulation | | | | | | | |
| 29 | PI*Z/PI*Q0 | 2 | PI*Without | 0 | 0 | 0 | N/A | N/A | N/A | Slightly |
| | clayton | | accumulation/PI* | | | | | | | at increased |
| | | | Severe | | | | | | | risk** |
| | | | accumulation | | | | | | | |
| 30 | PI*Z/PI*M | 2 | PI*Without | 0 | 0 | 0 | N/A | N/A | N/A | Slightly |
| | heerlen | | accumulation/PI* | | | | | | | at increased |
| | | | Severe | | | | | | | risk** |
| | | | accumulation | | | | | | | |
| 31 | PI*S/PI*I | 2 | PI*Mild | 0 | 0 | 0 | N/A | N/A | N/A | Unknown |
| | | | accumulation/PI* | | | | | | | risk |
| | | | Mild | | | | | | | |
| | | | accumulation | | | | | | | |
| 32 | PI*S/PI*M | 2 | PI*Without | 0 | 0 | 0 | N/A | N/A | N/A | Unknown |
| | procida | | accumulation/PI* | | | | | | | risk |
| | | | Mild | | | | | | | |
| | | | accumulation | | | | | | | |
| 33 | PI*S/PI*M | 2 | PI*Mild | 1 | 0 | 1 | 0.00% | 0.00% | 73.00% | Slightly |
| | malton | | accumulation/PI* | | | | | | | at increased |
| | | | Severe | | | | | | | risk** |
| | | | accumulation | | | | | | | |
| 34 | PI*S/PI*S | 2 | PI*Mild | 0 | 0 | 0 | N/A | N/A | N/A | Unknown |
| | iiyama | | accumulation/PI* | | | | | | | risk |
| | | | Severe | | | | | | | |
| | | | accumulation | | | | | | | |
| 35 | PI*S/PI*Q0 | 2 | PI*Without | 0 | 0 | 0 | N/A | N/A | N/A | Unknown |
| | granite falls | | accumulation/PI* | | | | | | | risk |
| | | | Mild | | | | | | | |
| | | | accumulation | | | | | | | |
| 36 | PI*S/PI*Q0 | 2 | PI*Without | 0 | 0 | 0 | N/A | N/A | N/A | Unknown |
| | west | | accumulation/PI* | | | | | | | risk |
| | | | Mild | | | | | | | |
| | | | accumulation | | | | | | | |
| 37 | PI*S/PI*Q0 | 2 | PI*Without | 0 | 0 | 0 | N/A | N/A | N/A | Unknown |
| | bellingham | | accumulation/PI* | | | | | | | risk |
| | | | Mild | | | | | | | |
| | | | accumulation | | | | | | | |
| 38 | PI*F/PI*S | 2 | PI*Without | 0 | 0 | 0 | N/A | N/A | N/A | Unknown |
| | | | accumulation/PI* | | | | | | | risk |
| | | | Mild | | | | | | | |
| | | | accumulation | | | | | | | |
| 39 | PI*S/PI*P | 2 | PI*Without | 0 | 0 | 0 | N/A | N/A | N/A | Unknown |
| | lowell | | accumulation/PI* | | | | | | | risk |
| | | | Mild | | | | | | | |
| | | | accumulation | | | | | | | |
| 40 | PI*S/PI*S | 2 | PI*Mild | 34 | 3 | 31 | 8.80% | 3.60% | 20.10% | Not likely |
| | | | accumulation/PI* | | | | | | | at increased |
| | | | Mild | | | | | | | risk |
| | | | accumulation | | | | | | | |
| 41 | PI*S/PI*Q0 | 2 | PI*Without | 0 | 0 | 0 | N/A | N/A | N/A | Unknown |
| | mattawa | | accumulation/PI* | | | | | | | risk |
| | | | Mild | | | | | | | |
| | | | accumulation | | | | | | | |
| 42 | PI*S/PI*Q0 | 2 | PI*Without | 2 | 1 | 1 | 50.00% | 12.10% | 87.90% | Unknown |
| | clayton | | accumulation/PI* | | | | | | | risk |
| | | | Mild | | | | | | | |
| | | | accumulation | | | | | | | |
| 43 | PI*S/PI*M | 2 | PI*Without | 0 | 0 | 0 | N/A | N/A | N/A | Unknown |
| | heerlen | | accumulation/PI* | | | | | | | risk |
| | | | Mild | | | | | | | |
| | | | accumulation | | | | | | | |
| 44 | PI*I/PI*I | 2 | PI*Mild | 0 | 0 | 0 | N/A | N/A | N/A | Unknown |
| | | | accumulation/PI* | | | | | | | risk |
| | | | Mild | | | | | | | |
| | | | accumulation | | | | | | | |
| 45 | PI*I/PI*M | 2 | PI*Without | 0 | 0 | 0 | N/A | N/A | N/A | Unknown |
| | procida | | accumulation/PI* | | | | | | | risk |
| | | | Mild | | | | | | | |
| | | | accumulation | | | | | | | |
| 46 | PI*I/PI*M | 2 | PI*Mild | 0 | 0 | 0 | N/A | N/A | N/A | Slightly |
| | malton | | accumulation/PI* | | | | | | | at increased |
| | | | Severe | | | | | | | risk** |
| | | | accumulation | | | | | | | |
| 47 | PI*I/PI*S | 2 | PI*Mild | 0 | 0 | 0 | N/A | N/A | N/A | Unknown |
| | iiyama | | accumulation/PI* | | | | | | | risk |
| | | | Severe | | | | | | | |
| | | | accumulation | | | | | | | |
| 48 | PI*I/PI*Q0 | 2 | PI*Without | 0 | 0 | 0 | N/A | N/A | N/A | Unknown |
| | granite falls | | accumulation/PI* | | | | | | | risk |
| | | | Mild | | | | | | | |
| | | | accumulation | | | | | | | |
| 49 | PI*I/PI*Q0 | 2 | PI*Without | 0 | 0 | 0 | N/A | N/A | V/A | Unknown |
| | west | | accumulation/PI* | | | | | | | risk |
| | | | Mild | | | | | | | |
| | | | accumulation | | | | | | | |
| 50 | PI*I/PI*Q0 | 2 | PI*Without | 0 | 0 | 0 | N/A | N/A | N/A | Unknown |
| | bellingham | | accumulation/PI* | | | | | | | risk |
| | | | Mild | | | | | | | |
| | | | accumulation | | | | | | | |
| 51 | PI*F/PI*I | 2 | PI*Without | 0 | 0 | 0 | N/A | N/A | N/A | Unknown |
| | | | accumulation/PI* | | | | | | | risk |
| | | | Mild | | | | | | | |
| | | | accumulation | | | | | | | |
| 52 | PI*I/PI*P | 2 | PI*Without | 0 | 0 | 0 | N/A | N/A | N/A | Unknown |
| | lowell | | accumulation/PI* | | | | | | | risk |
| | | | Mild | | | | | | | |
| | | | accumulation | | | | | | | |
| 53 | PI*I/PI*Q0 | 2 | PI*Without | 0 | 0 | 0 | N/A | N/A | N/A | Unknown |
| | mattawa | | accumulation/PI* | | | | | | | risk |
| | | | Mild | | | | | | | |
| | | | accumulation | | | | | | | |
| 54 | PI*I/PI*Q0 | 2 | PI*Without | 0 | 0 | 0 | N/A | N/A | N/A | Unknown |
| | clayton | | accumulation/PI* | | | | | | | risk |
| | | | Mild | | | | | | | |
| | | | accumulation | | | | | | | |
| 55 | PI*I/PI*M | 2 | PI*Without | 0 | 0 | 0 | N/A | N/A | N/A | Unknown |
| | heerlen | | accumulation/PI* | | | | | | | risk |
| | | | Mild | | | | | | | |
| | | | accumulation | | | | | | | |
| 56 | PI*F/PI*M | 2 | PI*Without | 0 | 0 | 0 | N/A | N/A | N/A | Not likely |
| | procida | | accumulation/PI* | | | | | | | at increased |
| | | | Without | | | | | | | risk** |
| | | | accumulation | | | | | | | |
| 57 | PI*F/PI*M | 2 | PI*Without | 0 | 0 | 0 | N/A | N/A | N/A | Slightly |
| | malton | | accumulation/PI* | | | | | | | at increased |
| | | | Severe | | | | | | | risk** |
| | | | accumulation | | | | | | | |
| 58 | PI*F/PI*S | 2 | PI*Without | 0 | 0 | 0 | N/A | N/A | N/A | Unknown |
| | iiyama | | accumulation/PI* | | | | | | | risk |
| | | | Severe | | | | | | | |
| | | | accumulation | | | | | | | |
| 59 | PI*F/PI*Q0 | 2 | PI*Without | 0 | 0 | 0 | N/A | N/A | N/A | Not likely |
| | granite falls | | accumulation/PI* | | | | | | | at increased |
| | | | Without | | | | | | | risk** |
| | | | accumulation | | | | | | | |
| 60 | PI*F/PI*Q0 | 2 | PI*Without | 0 | 0 | 0 | N/A | N/A | N/A | Not likely |
| | west | | accumulation/PI* | | | | | | | at increased |
| | | | Without | | | | | | | risk** |
| | | | accumulation | | | | | | | |
| 61 | PI*F/PI*Q0 | 2 | PI*Without | 0 | 0 | 0 | N/A | N/A | N/A | Not likely |
| | bellingham | | accumulation/PI* | | | | | | | at increased |
| | | | Without | | | | | | | risk** |
| | | | accumulation | | | | | | | |
| 62 | PI*F/PI*F | 2 | PI*Without | 1 | 0 | 1 | 0.00% | 0.00% | 73.00% | Not likely |
| | | | accumulation/PI* | | | | | | | at increased |
| | | | Without | | | | | | | risk** |
| | | | accumulation | | | | | | | |
| 63 | PI*F/PI*P | 2 | PI*Without | 0 | 0 | 0 | N/A | N/A | N/A | Not likely |
| | lowell | | accumulation/PI* | | | | | | | at increased |
| | | | Without | | | | | | | risk** |
| | | | accumulation | | | | | | | |
| 64 | PI*F/PI*Q0 | 2 | PI*Without | 0 | 0 | 0 | N/A | N/A | N/A | Not likely |
| | mattawa | | accumulation/PI* | | | | | | | at increased |
| | | | Without | | | | | | | risk** |
| | | | accumulation | | | | | | | |
| 65 | PI*F/PI*Q0 | 2 | PI*Without | 1 | 0 | 1 | 0.00% | 0.00% | 73.00% | Not likely |
| | clayton | | accumulation/PI* | | | | | | | at increased |
| | | | Without | | | | | | | risk** |
| | | | accumulation | | | | | | | |
| 66 | PI*F/PI*M | 2 | PI*Without | 0 | 0 | 0 | N/A | N/A | N/A | Not likely |
| | heerlen | | accumulation/PI* | | | | | | | at increased |
| | | | Without | | | | | | | risk** |
| | | | accumulation | | | | | | | |
| 67 | PI*M | 2 | PI*Without | 2 | 1 | 1 | 50.00% | 12.10% | 87.90% | Not likely |
| | procida/PI* | | accumulation/PI* | | | | | | | at increased |
| | M procida | | Without | | | | | | | risk** |
| | | | accumulation | | | | | | | |
| 68 | PI*M | 2 | PI*Without | 0 | 0 | 0 | N/A | N/A | N/A | Slightly |
| | malton/PI* | | accumulation/PI* | | | | | | | at increased |
| | M procida | | Severe | | | | | | | risk** |
| | | | accumulation | | | | | | | |
| 69 | PI*M | 2 | PI*Without | 0 | 0 | 0 | N/A | N/A | N/A | Unknown |
| | procida/PI* | | accumulation/PI* | | | | | | | risk |
| | S iiyama | | Severe | | | | | | | |
| | | | accumulation | | | | | | | |
| 70 | PI*M | 2 | PI*Without | 0 | 0 | 0 | N/A | N/A | N/A | Not likely |
| | procida/PI* | | accumulation/PI* | | | | | | | at increased |
| | Q0 granite | | Without | | | | | | | risk** |
| | falls | | accumulation | | | | | | | |
| 71 | PI*M | 2 | PI*Without | 0 | 0 | 0 | N/A | N/A | N/A | Not likely |
| | procida/PI* | | accumulation/PI* | | | | | | | at increased |
| | Q0 west | | Without | | | | | | | risk** |
| | | | accumulation | | | | | | | |
| 72 | PI*M | 2 | PI*Without | 0 | 0 | 0 | N/A | N/A | N/A | Not likely |
| | procida/PI* | | accumulation/PI* | | | | | | | at increased |
| | Q0 | | Without | | | | | | | risk** |
| | bellingham | | accumulation | | | | | | | |
| 73 | PI*M | 2 | PI*Without | 0 | 0 | 0 | N/A | N/A | N/A | Not likely |
| | procida/PI* | | accumulation/PI* | | | | | | | at increased |
| | P lowell | | Without | | | | | | | risk** |
| | | | accumulation | | | | | | | |
| 74 | PI*M | 2 | PI*Without | 0 | 0 | 0 | N/A | N/A | N/A | Not likely |
| | procida/PI* | | accumulation/PI* | | | | | | | at increased |
| | Q0 mattawa | | Without | | | | | | | risk** |
| | | | accumulation | | | | | | | |
| 75 | PI*M | 2 | PI*Without | 0 | 0 | 0 | N/A | N/A | N/A | Not likely |
| | procida/PI* | | accumulation/PI* | | | | | | | at increased |
| | Q0 clayton | | Without | | | | | | | risk** |
| | | | accumulation | | | | | | | |
| 76 | PI*M | 2 | PI*Without | 0 | 0 | 0 | N/A | N/A | N/A | Not likely |
| | procida/PI* | | accumulation/PI* | | | | | | | at increased |
| | M heerlen | | Without | | | | | | | risk** |
| | | | accumulation | | | | | | | |
| 77 | PI*M | 2 | PI*Severe | 16 | 8 | 8 | 50.00% | 31.00% | 69.00% | Slightly |
| | malton/PI* | | accumulation/PI* | | | | | | | at increased |
| | M malton | | Severe | | | | | | | risk |
| | | | accumulation | | | | | | | |
| 78 | PI*M | 2 | PI*Severe | 0 | 0 | 0 | N/A | N/A | N/A | Slightly |
| | malton/PI*S | | accumulation/PI* | | | | | | | at increased |
| | iiyama | | Severe | | | | | | | risk** |
| | | | accumulation | | | | | | | |
| 79 | PI*M | 2 | PI*Without | 0 | 0 | 0 | N/A | N/A | N/A | Slightly |
| | malton/PI*Q0 | | accumulation/PI* | | | | | | | at increased |
| | granite | | Severe | | | | | | | risk** |
| | falls | | accumulation | | | | | | | |
| 80 | PI*M | 2 | PI*Without | 0 | 0 | 0 | N/A | N/A | N/A | Slightly |
| | malton/PI*Q0 | | accumulation/PI* | | | | | | | at increased |
| | west | | Severe | | | | | | | risk** |
| | | | accumulation | | | | | | | |
| 81 | PI*M | 2 | PI*Without | 0 | 0 | 0 | N/A | N/A | N/A | Slightly |
| | malton/PI*Q0 | | accumulation/PI* | | | | | | | at increased |
| | bellingham | | Severe | | | | | | | risk** |
| | | | accumulation | | | | | | | |
| 82 | PI*M | 2 | PI*Without | 0 | 0 | 0 | N/A | N/A | N/A | Slightly |
| | malton/PI*P | | accumulation/PI* | | | | | | | at increased |
| | lowell | | Severe | | | | | | | risk** |
| | | | accumulation | | | | | | | |
| 83 | PI*M | 2 | PI*Without | 2 | 0 | 2 | 0.00% | 0.00% | 57.50% | Slightly |
| | malton/PI*Q0 | | accumulation/PI* | | | | | | | at increased |
| | mattawa | | Severe | | | | | | | risk** |
| | | | accumulation | | | | | | | |
| 84 | PI*M | 2 | PI*Without | 0 | 0 | 0 | N/A | N/A | N/A | Slightly |
| | malton/PI*Q0 | | accumulation/PI* | | | | | | | at increased |
| | clayton | | Severe | | | | | | | risk** |
| | | | accumulation | | | | | | | |
| 85 | PI*M | 2 | PI*Without | 0 | 0 | 0 | N/A | N/A | N/A | Slightly |
| | malton/PI* | | accumulation/PI* | | | | | | | at increased |
| | M heerlen | | Severe | | | | | | | risk** |
| | | | accumulation | | | | | | | |
| 86 | PI*S | 2 | PI*Severe | 2 | 0 | 2 | 0.00% | 0.00% | 57.50% | Unknown |
| | iiyama/PI*S | | accumulation/PI* | | | | | | | risk |
| | iiyama | | Severe | | | | | | | |
| | | | accumulation | | | | | | | |
| 87 | PI*Q0 | 2 | PI*Without | 0 | 0 | 0 | N/A | N/A | N/A | Unknown |
| | granite | | accumulation/PI* | | | | | | | risk |
| | falls/PI*S | | Severe | | | | | | | |
| | iiyama | | accumulation | | | | | | | |
| 88 | PI*S | 2 | PI*Without | 0 | 0 | 0 | N/A | N/A | N/A | Unknown |
| | iiyama/PI*Q0 | | accumulation/PI* | | | | | | | risk |
| | west | | Severe | | | | | | | |
| | | | accumulation | | | | | | | |
| 89 | PI*S | 2 | PI*Without | 0 | 0 | 0 | N/A | N/A | N/A | Unknown |
| | iiyama/PI*Q | | accumulation/PI* | | | | | | | risk |
| | 0 | | Severe | | | | | | | |
| | bellingham | | accumulation | | | | | | | |
| 90 | PI*P | 2 | PI*Without | 0 | 0 | 0 | N/A | N/A | N/A | Unknown |
| | lowell/PI*S | | accumulation/PI* | | | | | | | risk |
| | iiyama | | Severe | | | | | | | |
| | | | accumulation | | | | | | | |
| 91 | PI*S | 2 | PI*Without | 0 | 0 | 0 | N/A | N/A | N/A | Unknown |
| | iiyama/PI*Q0 | | accumulation/PI* | | | | | | | risk |
| | mattawa | | Severe | | | | | | | |
| | | | accumulation | | | | | | | |
| 92 | PI*S | 2 | PI*Without | 2 | 0 | 2 | 0.00% | 0.00% | 57.50% | Unknown |
| | iiyama/PI*Q0 | | accumulation/PI* | | | | | | | risk |
| | clayton | | Severe | | | | | | | |
| | | | accumulation | | | | | | | |
| 93 | PI*M | 2 | PI*Without | 0 | 0 | 0 | N/A | N/A | N/A | Unknown |
| | heerlen/PI*S | | accumulation/PI* | | | | | | | risk |
| | iiyama | | Severe | | | | | | | |
| | | | accumulation | | | | | | | |
| 94 | PI*Q0 | 2 | PI*Without | 0 | 0 | 0 | N/A | N/A | N/A | Not likely |
| | granite | | accumulation/PI* | | | | | | | at increased |
| | falls/PI*Q0 | | Without | | | | | | | risk** |
| | granite falls | | accumulation | | | | | | | |
| 95 | PI*Q0 | 2 | PI*Without | 0 | 0 | 0 | N/A | N/A | N/A | Not likely |
| | granite | | accumulation/PI* | | | | | | | at increased |
| | falls/PI*Q0 | | Without | | | | | | | risk** |
| | west | | accumulation | | | | | | | |
| 96 | PI*Q0 | 2 | PI*Without | 0 | 0 | 0 | N/A | N/A | N/A | Not likely |
| | bellingham/ | | accumulation/PI* | | | | | | | at increased |
| | PI*Q0 | | Without | | | | | | | risk** |
| | granite falls | | accumulation | | | | | | | |
| 97 | PI*P | 2 | PI*Without | 0 | 0 | 0 | N/A | N/A | N/A | Not likely |
| | lowell/PI*Q0 | | accumulation/PI* | | | | | | | at increased |
| | granite | | Without | | | | | | | risk** |
| | falls | | accumulation | | | | | | | |
| 98 | PI*Q0 | 2 | PI*Without | 0 | 0 | 0 | N/A | N/A | N/A | Not likely |
| | granite | | accumulation/PI* | | | | | | | at increased |
| | falls/PI*Q0 | | Without | | | | | | | risk** |
| | mattawa | | accumulation | | | | | | | |
| 99 | PI*Q0 | 2 | PI*Without | 0 | 0 | 0 | N/A | N/A | N/A | Not likely |
| | clayton/PI* | | accumulation/PI* | | | | | | | at increased |
| | Q0 granite | | Without | | | | | | | risk** |
| | falls | | accumulation | | | | | | | |
| 100 | PI*M | 2 | PI*Without | 1 | 0 | 1 | 0.00% | 0.00% | 73.00% | Not likely |
| | heerlen/PI* | | accumulation/PI* | | | | | | | at increased |
| | Q0 granite | | Without | | | | | | | risk** |
| | falls | | accumulation | | | | | | | |
| 101 | PI*Q0 | 2 | PI*Without | 0 | 0 | 0 | N/A | N/A | N/A | Not likely |
| | west/PI*Q0 | | accumulation/PI* | | | | | | | at increased |
| | west | | Without | | | | | | | risk** |
| | | | accumulation | | | | | | | |
| 102 | PI*Q0 | 2 | PI*Without | 0 | 0 | 0 | N/A | N/A | N/A | Not likely |
| | bellingham/ | | accumulation/PI* | | | | | | | at increased |
| | PI*Q0 west | | Without | | | | | | | risk** |
| | | | accumulation | | | | | | | |
| 103 | PI*P | 2 | PI*Without | 0 | 0 | 0 | N/A | N/A | N/A | Not likely |
| | lowell/PI*Q0 | | accumulation/PI* | | | | | | | at increased |
| | west | | Without | | | | | | | risk** |
| | | | accumulation | | | | | | | |
| 104 | PI*Q0 | 2 | PI*Without | 0 | 0 | 0 | N/A | N/A | N/A | Not likely |
| | mattawa/PI* | | accumulation/PI* | | | | | | | at increased |
| | Q0 west | | Without | | | | | | | risk** |
| | | | accumulation | | | | | | | |
| 105 | PI*Q0 | 2 | PI*Without | 0 | 0 | 0 | N/A | N/A | N/A | Not likely |
| | clayton/PI* | | accumulation/PI* | | | | | | | at increased |
| | Q0 west | | Without | | | | | | | risk** |
| | | | accumulation | | | | | | | |
| 106 | PI*M | 2 | PI*Without | 0 | 0 | 0 | N/A | N/A | N/A | Not likely |
| | heerlen/PI* | | accumulation/PI* | | | | | | | at increased |
| | Q0 west | | Without | | | | | | | risk** |
| | | | accumulation | | | | | | | |
| 107 | PI*Q0 | 2 | PI*Without | 2 | 0 | 2 | 0.00% | 0.00% | 57.50% | Not likely |
| | bellingham/ | | accumulation/PI* | | | | | | | at increased |
| | PI*Q0 | | Without | | | | | | | risk** |
| | bellingham | | accumulation | | | | | | | |
| 108 | PI*P | 2 | PI*Without | 0 | 0 | 0 | N/A | N/A | N/A | Not likely |
| | lowell/PI*Q0 | | accumulation/PI* | | | | | | | at increased |
| | bellingham | | Without | | | | | | | risk** |
| | | | accumulation | | | | | | | |
| 109 | PI*Q0 | 2 | PI*Without | 0 | 0 | 0 | N/A | N/A | N/A | Not likely |
| | bellingham/ | | accumulation/PI* | | | | | | | at increased |
| | PI*Q0 | | Without | | | | | | | risk** |
| | mattawa | | accumulation | | | | | | | |
| 110 | PI*Q0 | 2 | PI*Without | 0 | 0 | 0 | N/A | N/A | N/A | Not likely |
| | bellingham/ | | accumulation/PI* | | | | | | | at increased |
| | PI*Q0 | | Without | | | | | | | risk** |
| | clayton | | accumulation | | | | | | | |
| 111 | PI*M | 2 | PI*Without | 0 | 0 | 0 | N/A | N/A | N/A | Not likely |
| | heerlen/PI* | | accumulation/PI* | | | | | | | at increased |
| | Q0 | | Without | | | | | | | risk** |
| | bellingham | | accumulation | | | | | | | |
| 112 | PI*P | 2 | PI*Without | 0 | 0 | 0 | N/A | N/A | N/A | Not likely |
| | lowell/PI*P | | accumulation/PI* | | | | | | | at increased |
| | lowell | | Without | | | | | | | risk** |
| | | | accumulation | | | | | | | |
| 113 | PI*P | 2 | PI*Without | 0 | 0 | 0 | N/A | N/A | N/A | Not likely |
| | lowell/PI*Q0 | | accumulation/PI* | | | | | | | at increased |
| | mattawa | | Without | | | | | | | risk** |
| | | | accumulation | | | | | | | |
| 114 | PI*P | 2 | PI*Without | 0 | 0 | 0 | N/A | N/A | N/A | Not likely |
| | lowell/PI*Q0 | | accumulation/PI* | | | | | | | at increased |
| | clayton | | Without | | | | | | | risk** |
| | | | accumulation | | | | | | | |
| 115 | PI*P | 2 | PI*Without | 0 | 0 | 0 | N/A | N/A | N/A | Not likely |
| | lowell/PI*M | | accumulation/PI* | | | | | | | at increased |
| | heerlen | | Without | | | | | | | risk** |
| | | | accumulation | | | | | | | |
| 116 | PI*Q0 | 2 | PI*Without | 2 | 0 | 2 | 0.00% | 0.00% | 57.50% | Not likely |
| | mattawa/PI* | | accumulation/PI* | | | | | | | at increased |
| | Q0 mattawa | | Without | | | | | | | risk** |
| | | | accumulation | | | | | | | |
| 117 | PI*Q0 | 2 | PI*Without | 0 | 0 | 0 | N/A | N/A | N/A | Not likely |
| | clayton/PI* | | accumulation/PI* | | | | | | | at increased |
| | Q0 mattawa | | Without | | | | | | | risk** |
| | | | accumulation | | | | | | | |
| 118 | PI*M | 2 | PI*Without | 0 | 0 | 0 | N/A | N/A | N/A | Not likely |
| | heerlen/PI* | | accumulation/PI* | | | | | | | at increased |
| | Q0 mattawa | | Without | | | | | | | risk** |
| | | | accumulation | | | | | | | |
| 119 | PI*Q0 | 2 | PI*Without | 0 | 0 | 0 | N/A | N/A | N/A | Not likely |
| | clayton/PI* | | accumulation/PI* | | | | | | | at increased |
| | Q0 clayton | | Without | | | | | | | risk** |
| | | | accumulation | | | | | | | |
| 120 | PI*M | 2 | PI*Without | 0 | 0 | 0 | N/A | N/A | N/A | Not likely |
| | heerlen/PI* | | accumulation/PI* | | | | | | | at increased |
| | Q0 clayton | | Without | | | | | | | risk** |
| | | | accumulation | | | | | | | |
| 121 | PI*M | 2 | PI*Without | 1 | 0 | 1 | 0.00% | 0.00% | 73.00% | Not likely |
| | heerlen/PI* | | accumulation/PI* | | | | | | | at increased |
| | M heerlen | | Without | | | | | | | risk** |
| | | | accumulation | | | | | | | |

***NOTE:
Without liver accumulation variants (PI*Without): PI*M, PI*M procida, PI*Q0 granite falls, PI*Q0 west, PI*Q0 bellingham, PI*F, PI*P lowell, PI*Q0 mattawa, PI*Q0 clayton, and PI*M heerlen; Mild liver accumulation variants (PI*Mild): PI*I, PI*S; Severe liver accumulation variants (PI*Severe): PI*Z, PI*S iiyama, PI*M malton.
**NOTE:
Exceptions to the general classification: Individuals with at least one PI*Z or PI*M malton severe accumulation variants and their combinations will be reported as “Slightly Increased risk”; All the combinations including two non-accumulation alleles will be reported as “Not likely at Increased risk”. Non-accumulation alleles: PI*M, PI*M procida, PI*Q0 granite falls, PI*Q0 west, PI*Q0 bellingham, PI*F, PI*P lowell, PI*Q0 mattawa, PI*Q0 clayton, and PI*M heerlen.

In some implementations, the one or more clinically relevant allelic variant genotypes comprises one or more variants of a gene, wherein the gene is a Coagulation Factor V (FV) gene, Coagulation Factor II (FII) gene, Serpin family C member 1 (SERPINC1) gene, protein S (PROS1) gene, or protein C (PROC) gene; and wherein the disease is a thrombophilia associated disease. In some aspects, the one or more clinically relevant allelic variants is selected from the group consisting of: (i) R506Q of the FV gene, (ii) FV Leiden of the FV gene, (iii) G20210A of the FII gene, (iv) A384S (G1246T) (rs121909548) of the SERPINC1 gene, (v) Intronic (rs2227589) of the SERPIN1C gene, (vi) Val30Glu (rs2227624) of the SERPIN1C gene, (vii) K196E of the PROS1 gene, (vii) K155E of the PROS1 gene, (viii) c.574_576dup/del (rs199469469) of the PROC gene, (ix) R147W of the PROC gene, (x) Arg42Cys of the PROC gene, (xi) Arg51Cys of the PROC gene, (xii) Val76Met of the PROC gene, (xiii) C 2633 G of the PROC gene, (xiv) C 2730 T of the PROC gene, (xv) G 3310 A of the PROC gene, (xvi) c.565C.T (p.Arg189Trp) of the PROC gene, (xvii) 2405C/T of the PROC gene, (xviii) 2418A/G of the PROC gene, (xix) C/T at −1654, A/G at −1641, A/T at −1476 of the PROC gene, and (xx) promoter GC haplotype of the PROC gene.

As another specific example, Table 6 below corresponds to an implementation in which the user's risk category, as described herein, for developing thrombophilia disease depends on the number of clinically reported cases of the respective one or more alleles, the percentage of clinically reported cases of the respective one or more alleles in which lung disease occurred, and the risk-based confidence interval.

TABLE 6
General Criteria for Thrombophilia Disease Risk Categorization

| Number of | % Reported | | |
|---|---|---|---|
| reported | clinical cases | | Reported Thrombophilia |
| clinical cases | with lung disease | 95% CI | disease risk category |
| n < 3 | Any | Any | Unknown risk |
| n ≥ 3 | <20% | 0-80% | Not Likely at |
| | | | increased risk |
| | | 0-100% | Unknown risk |
| | 20-80% | 0-80% | Not Likely at |
| | | | increased risk |
| | | 20-100% | Slightly increased risk |
| | | 0-100% | Unknown risk |
| | 80% | 0-100% | Unknown risk |
| | | 20-100% | Slightly increased risk |
| | | >80% | Increased risk |

As another specific example, Table 7 below corresponds to an implementation in which the disease is a thrombophilia associated disease, and the allele(s) of the user are selected from one or more clinically relevant allelic variant genotypes of a gene. The gene may be an FV gene, an FII gene, a SERPINC11 gene, a PROS1 gene, or a PROC gene.

TABLE 7

| | | | | | | % | | |
|---|---|---|---|---|---|---|---|---|
| | | Clinically Relevant | | | | Individuals | | |
| | | Allelic Variant | | | Reported | with one or | | |
| | | Genotypes Detected | Users with | Users | Thrombophilia | more | | |
| | | in Genomic DNA of | Variant & | with | Disease Risk | Variants & | | |
| # | Gene | the User | Thrombophilia | Variant | Category | Thrombophilia | CI 95% | |
| 1 | FV | R506Q or FV | 20002 | 3756 | Increased risk | 84.2% | 83.8% | 84.6% |
| | | Leiden | | | | | | |
| 2 | FII | G20210A | 20524 | 1397 | Increased risk | 93.6% | 93.4% | 93.9% |
| 3 | SERPINC1 | A384S (G1246T) | 39 | 17 | Slightly | 69.6% | 58.8% | 78.7% |
| | | (rs121909548) | | | increased risk | | | |
| 4 | SERPINC1 | Intronic | 701 | 902 | Slightly | 43.7% | 41.7% | 45.8% |
| | | (rs2227589) | | | increased risk | | | |
| 5 | SERPINC1 | Val30Glu | 11 | 7 | Slightly | 61.1% | 42.0% | 77.3% |
| | | (rs2227624) | | | increased risk | | | |
| 6 | PROS1 | K196E | 18 | 66 | Not Likely at | 21.4% | 15.0% | 29.6% |
| | | | | | Increased risk | | | |
| 7 | PROS1 | K155E | 6 | 3 | Slightly | 66.7% | 39.8% | 85.8% |
| | | | | | increased risk | | | |
| 8 | PROC | c.574_576dup/del | 67 | 25 | Slightly | 72.8% | 64.6% | 79.7% |
| | | (rs199469469) | | | increased risk | | | |
| 9 | PROC | R147W | 23 | 41 | Slightly | 35.9% | 26.8% | 46.2% |
| | | | | | increased risk | | | |
| 10 | PROC | Arg42Cys | 1 | 0 | Unknown risk | 100.0% | 27.0% | 100.0% |
| 11 | PROC | Arg51Cys | 1 | 0 | Unknown risk | 100.0% | 27.0% | 100.0% |
| 12 | PROC | Val76Met | 1 | 0 | Unknown risk | 100.0% | 27.0% | 100.0% |
| 13 | PROC | C 2633 G | 1 | 0 | Unknown risk | 100.0% | 27.0% | 100.0% |
| 14 | PROC | C 2730 T | 1 | 0 | Unknown risk | 100.0% | 27.0% | 100.0% |
| 15 | PROC | G 3310 A | 223 | 232 | Slightly | 49.0% | 45.2% | 52.9% |
| | | | | | increased risk | | | |
| 16 | PROC | c.565C.T | 59 | 9 | Slightly | 86.8% | 78.6% | 92.1% |
| | | (p.Arg189Trp) | | | increased risk | | | |
| 17 | PROC | 2405C/T | 2456 | 2896 | Slightly | 45.9% | 44.8% | 47.0% |
| | | | | | increased risk | | | |
| 18 | PROC | 2418A/G | 3070 | 3316 | Slightly | 48.1% | 47.0% | 49.1% |
| | | | | | increased risk | | | |
| 19 | PROC | C/T at −1654, A/G | 213 | 257 | Slightly | 45.3% | 41.6% | 49.1% |
| | | at −1641, and A/T | | | increased risk | | | |
| | | at −1476 | | | | | | |
| 20 | PROC | promoter GC | 22 | 56 | Slightly | 28.2% | 20.7% | 37.2% |
| | | haplotype | | | increased risk | | | |

### Additional Considerations

Although the disclosure herein sets forth a detailed description of numerous different aspects, it should be understood that the legal scope of the description is defined by the words of the claims set forth at the end of this patent and equivalents. The detailed description is to be construed as exemplary only and does not describe every possible aspect since describing every possible aspect would be impractical. Numerous alternative aspects may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain aspects are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example aspects, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example aspects, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example aspects, the processor or processors may be located in a single location, while in other aspects the processors may be distributed across a number of locations.

The performance of certain of the operations may be distributed among the one or more processors, not only residing within a single machine, but deployed across a number of machines. In some example aspects, the one or more processors or processor-implemented modules may be located in a single geographic location (e.g., within a home environment, an office environment, or a server farm). In other aspects, the one or more processors or processor-implemented modules may be distributed across a number of geographic locations.

This detailed description is to be construed as exemplary only and does not describe every possible aspect, as describing every possible aspect would be impractical, if not impossible. A person of ordinary skill in the art may implement numerous alternate aspects, using either current technology or technology developed after the filing date of this application.

Those of ordinary skill in the art will recognize that a wide variety of modifications, alterations, and combinations can be made with respect to the above described aspects without departing from the scope of the invention, and that such modifications, alterations, and combinations are to be viewed as being within the ambit of the inventive concept.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

What is claimed is:

1. A genetic-based biological sample analysis system configured to detect a user-specific genetic health risk related to a disease, the genetic-based biological sample analysis system comprising:
a server comprising one or more processors and server computing instructions configured for execution by the one or more processors of the server, the server communicatively coupled to a computer network;
a genetic risk model stored in a memory communicatively coupled to the server and accessible by the one or more processors of the server, the genetic risk model configured to output a classification defining respective risks of respective users developing the disease,
wherein the classification as output by the genetic risk model is selected from a predetermined set of risk categories comprising: (a) an Increased Risk Category, (b) a Slightly Increased Risk Category, (c) a Not Likely at Increased Risk Category, and (d) an Unknown Risk Category,
wherein the risk categories are ordinal with respect to one another ordered based on respective percentage risk values or ranges of contracting the disease,
wherein the Increased Risk Category is assigned an upper percentage risk value or range,
wherein the Not Likely at Increased Risk Category is assigned a lower percentage risk value or range,
wherein the Slightly Increased Risk Category comprises a middle percentage risk value or range that is less than the upper percentage risk value or range but greater than the lower percentage risk value or range, and
wherein the Unknown Risk Category has an undetermined percentage risk value or range; and
a user application (app) implementing a user interface and comprising computing instructions configured for execution on a computing device, the user app configured to send to and receive data from the server,
wherein the server computing instructions, when executed by the one or more processors of the server, cause the one or more processors of the server to:
obtain, from a display of the computing device, user specific data of a user for detecting a likelihood of occurrence of a user-specific disease,
generate a profile of the user comprising the user specific data, wherein generation of the profile causes a user test kit to be delivered to the user, wherein the user test kit is configured to collect a biological sample of the user, the biological sample comprising genomic deoxyribonucleic acid (DNA) of the user as extracted from the biological sample,
receive lab-based genetic analysis output based on the genomic DNA of the user, wherein generation of the lab-based genetic analysis output comprises determination of one or more alleles of the user selected from one or more clinically relevant allelic variant genotypes,
output a classification for the user, based on the lab-based genetic analysis output, by the genetic risk model, the classification defining a user-specific risk of the user to develop the disease based on the one or more alleles as selected for the user,
generate a user-specific genetic health risk determination for the user based on the classification of the user and the lab-based genetic analysis output, and
transmit, to the user app, the user-specific genetic health risk determination for display on the user interface.

2. The genetic-based biological sample analysis system 1, wherein the classification as output for the user comprises the Unknown Risk Category, the classification output based upon detecting at least one of: (i) the one or more clinically relevant allelic variant genotypes as selected for the user are associated with two or less clinically reported cases, or (ii) the one or more clinically relevant allelic variant genotypes as selected for the user are associated with three or more clinically reported cases but the undetermined percentage risk value or range overlaps with at least one of the upper percentage risk value or range, the middle percentage risk value or range, or the lower percentage risk value or range.

3. The genetic-based biological sample analysis system 1, wherein the classification as output for the user comprises the Not Likely at Increased Risk category, the classification output based upon detecting that the one or more clinically relevant allelic variant genotypes as selected for the user are associated with three or more clinically reported cases, wherein the lower percentage risk value or range is at least one of: (i) less than a maximum percent as defined by clinically reported cases of the respective one or more alleles associated with a not likely increased risk of developing the disease, and for a respective risk-based confidence interval, or (ii) between the maximum percent as defined by clinically reported cases of the respective one or more alleles associated with the not likely increased risk of developing the disease and a minimum percent as defined by the clinically reported cases of the respective one or more alleles associated with an increased risk of developing the disease, and for such respective risk-based confidence interval.

4. The genetic-based biological sample analysis system 1, wherein the classification as output for the user comprises the Slightly Increased Risk category, the classification output based upon detecting that the one or more clinically relevant allelic variant genotypes as selected for the user are associated with three or more clinically reported cases, wherein the middle percentage risk value or range is at least one of: (i) more than a minimum percent as defined by clinically reported cases of the respective one or more alleles associated with an increased risk of developing the disease, and for a respective risk-based confidence interval, or (ii) between a maximum percent as defined by clinically reported cases of the respective one or more alleles associated with the not likely increased risk of developing the disease and the minimum percent as defined the clinically reported cases of the respective one or more alleles associated with an increased risk of developing the disease, and for such respective risk-based confidence interval.

5. The genetic-based biological sample analysis system 1, wherein the classification as output for the user comprises the Increased Risk category, the classification output based upon detecting that the one or more clinically relevant allelic variant genotypes as selected for the user are associated with three or more clinically reported cases, wherein the upper percentage risk value or range is more than a minimum percent defined clinically reported cases of the respective one or more alleles associated with an increased risk of developing the disease, and for a respective risk-based confidence interval.

6. The genetic-based biological sample analysis system of claim 1, wherein the one or more alleles of the user are selected from one or more clinically relevant allelic variants of the serpin peptidase inhibitor class A member 1 (SERPINA1) gene, and wherein the disease is lung disease associated with alpha1-antitrypsin deficiency (A1ATD) and/or liver disease associated with alpha1-antitrypsin deficiency (A1ATD).

7. The genetic-based biological sample analysis system of claim 6, wherein the classification as output for the user comprises the Not Likely at Increased Risk category, the classification output based upon detecting at least one of: (i) the one or more clinically relevant allelic variant genotypes as selected for the user are associated with three or more clinically reported cases, the lower percentage risk value or range is less than 20 percent defining lung or liver disease occurring in less than 20 percent of clinically reported cases of the respective one or more alleles, and a respective risk-based confidence interval between zero to 80 percent, or (ii) the one or more clinically relevant allelic variant genotypes as selected for the user are associated with three or more clinically reported cases for which lung or liver disease occurred in 20 to 80 percent of clinically reported cases of the respective one or more alleles, and a respective risk-based confidence interval between zero to 80 percent.

8. The genetic-based biological sample analysis system of claim 6, wherein the classification as output for the user comprises the Slightly Increased Risk category, the classification output based upon detecting at least one of: (i) the one or more clinically relevant allelic variant genotypes as selected for the user are associated with three or more clinically reported cases, the middle percentage risk value or range is between 20 percent and 80 percent defining lung or liver disease occurring in 20 to 80 percent of clinically reported cases of the respective one or more alleles, and a respective risk-based confidence interval between 20 to 100 percent, or (ii) the one or more clinically relevant allelic variant genotypes as selected for the user are associated with three or more clinically reported cases, lung or liver disease occurred in at least 80 percent of clinically reported cases of the respective one or more alleles, and a respective risk-based confidence interval between 20 to 100 percent.

9. The genetic-based biological sample analysis system of claim 6, wherein the classification as output for the user comprises the Increased Risk category, the classification output based upon detecting the one or more clinically relevant allelic variant genotypes as selected for the user are associated with three or more clinically reported cases, the upper percentage risk value or range is greater than 80 percent defining lung or liver disease occurring in at least 80 percent of clinically reported cases of the respective one or more alleles, and a respective risk-based confidence interval greater than 80 percent.

10. The genetic-based biological sample analysis system of claim 1, wherein the lab-based genetic analysis output further comprises one or more of: (a) a quantity of clinically reported cases of the respective one or more alleles, (b) a percentage of clinically reported cases of the respective one or more alleles in which the disease occurred, and/or (c) a respective risk-based confidence interval.

11. The genetic-based biological sample analysis system of claim 1, wherein the user-specific genetic health risk determination may comprise a report selected from a preset list of report types based on the one or more clinically relevant allelic variant genotypes.

12. The genetic-based biological sample analysis system of claim 6, wherein the one or more variants of the SERPINA1 gene include one or more of: (i) PI*S; (ii) PI*Z; (iii) PI*I; (iv) PI*M procida; (v) PI*M malton; (vi) PI*S iiyama; (vii) PI*Q0 granite falls; (viii) PI*Q0 west; (ix) PI*Q0 bellingham; (x) PI*F; (xi) PI*P lowell; (xii) PI*Q0 mattawa; (xiii) PI*Q0 clayton, and (xiv) PI*M Heerlen.

13. The genetic-based biological sample analysis system 1, wherein the one or more clinically relevant allelic variant genotypes comprises one or more variants of a gene, wherein the gene is a Coagulation Factor V (FV) gene, Coagulation Factor II (FII) gene, Serpin family C member 1 (SERPINC1) gene, protein S (PROS1) gene, or protein C (PROC) gene; and wherein the disease is a thrombophilia associated disease.

14. The genetic-based biological sample analysis system of claim 13, wherein the one or more clinically relevant allelic variants is selected from the group consisting of: (i) R506Q of the FV gene, (ii) FV Leiden of the FV gene, (iii) G20210A of the FII gene, (iv) A384S (G1246T) (rs121909548) of the SERPINC1 gene, (v) Intronic (rs2227589) of the SERPIN1C gene, (vi) Val30Glu (rs2227624) of the SERPIN1C gene, (vii) K196E of the PROS1 gene, (vii) K155E of the PROS1 gene, (viii) c.574_576dup/del (rs199469469) of the PROC gene, (ix) R147W of the PROC gene, (x) Arg42Cys of the PROC gene, (xi) Arg51Cys of the PROC gene, (xii) Val76Met of the PROC gene, (xiii) C 2633 G of the PROC gene, (xiv) C 2730 T of the PROC gene, (xv) G 3310 A of the PROC gene, (xvi) c.565C.T (p.Arg189Trp) of the PROC gene, (xvii) 2405C/T of the PROC gene, (xviii) 2418A/G of the PROC gene, (xix) C/T at −1654, A/G at −1641, A/T at −1476 of the PROC gene, and (xx) promoter GC haplotype of the PROC gene.

15. The genetic-based biological sample analysis system of claim 13, wherein the classification as output for the user comprises the Not Likely at Increased Risk category, the classification output based upon detecting at least one of: (i) the one or more clinically relevant allelic variant genotypes as selected for the user are associated with three or more clinically reported cases, the lower percentage risk value or range is less than 20 percent defining lung or liver disease occurring in less than 20 percent of clinically reported cases of the respective one or more alleles, and a respective risk-based confidence interval between zero to 80 percent, or (ii) the one or more clinically relevant allelic variant genotypes as selected for the user are associated with three or more clinically reported cases for which lung or liver disease occurred in 20 to 80 percent of clinically reported cases of the respective one or more alleles, and a respective risk-based confidence interval between zero to 80 percent.

16. The genetic-based biological sample analysis system of claim 13, wherein the classification as output for the user comprises the Slightly Increased Risk category, the classification output based upon detecting at least one of: (i) the one or more clinically relevant allelic variant genotypes as selected for the user are associated with three or more clinically reported cases, the middle percentage risk value or range is between 20 percent and 80 percent defining lung or liver disease occurring in 20 to 80 percent of clinically reported cases of the respective one or more alleles, and a respective risk-based confidence interval between 20 to 100 percent, or (ii) the one or more clinically relevant allelic variant genotypes as selected for the user are associated with three or more clinically reported cases, lung or liver disease occurred in at least 80 percent of clinically reported cases of the respective one or more alleles, and a respective risk-based confidence interval between 20 to 100 percent.

17. The genetic-based biological sample analysis system of claim 13, wherein the classification as output for the user comprises the Increased Risk category, the classification output based upon detecting the one or more clinically relevant allelic variant genotypes as selected for the user are associated with three or more clinically reported cases, the upper percentage risk value or range is greater than 80 percent defining lung or liver disease occurring in at least 80 percent of clinically reported cases of the respective one or more alleles, and a respective risk-based confidence interval greater than 80 percent.

18. A genetic-based biological sample analysis method for detecting a user-specific genetic health risk related to a disease, the genetic-based biological sample analysis method comprising:
obtaining, by one or more processors, user specific data of a user for detecting a likelihood of occurrence of a user-specific disease;
generating, by the one or more processors, a profile of the user comprising the user specific data, wherein generation of the profile causes a user test kit to be delivered to the user, wherein the user test kit is configured to collect a biological sample of the user, the biological sample comprising genomic deoxyribonucleic acid (DNA) of the user as extracted from the biological sample;
receiving, at the one or more processors, lab-based genetic analysis output based on the genomic DNA of the user, wherein generation of the lab-based genetic analysis output comprises determination of one or more alleles of the user selected from one or more clinically relevant allelic variant genotypes;
outputting, by the one or more processors, a classification for the user, based on the lab-based genetic analysis output, by a genetic risk model, the classification defining a user-specific risk of the user to develop the disease based on the one or more alleles as selected for the user,
wherein the genetic risk model is configured to output a classification defining respective risks of respective users developing the disease,
wherein the classification as output by the genetic risk model is selected from a predetermined set of risk categories comprising: (a) an Increased Risk Category, (b) a Slightly Increased Risk Category, (c) a Not Likely at Increased Risk Category, and (d) an Unknown Risk Category,
wherein the risk categories are ordinal with respect to one another ordered based on respective percentage risk values or ranges of contracting the disease,
wherein the Increased Risk Category is assigned an upper percentage risk value or range,
wherein the Not Likely at Increased Risk Category is assigned a lower percentage risk value or range,
wherein the Slightly Increased Risk Category comprises a middle percentage risk value or range that is less than the upper percentage risk value or range but greater than the lower percentage risk value or range, and
wherein the Unknown Risk Category has an undetermined percentage risk value or range;
generating, by the one or more processors, a user-specific genetic health risk determination for the user based on the classification of the user and the lab-based genetic analysis output; and
providing, by the one or more processors, to the user the user-specific genetic health risk determination.

19. The genetic-based biological sample analysis method of claim 18, wherein the classification as output for the user comprises the Unknown Risk Category, the classification output based upon detecting at least one of: (i) the one or more clinically relevant allelic variant genotypes as selected for the user are associated with two or less clinically reported cases, or (ii) the one or more clinically relevant allelic variant genotypes as selected for the user are associated with three or more clinically reported cases but the undetermined percentage risk value or range overlaps with at least one of the upper percentage risk value or range, the middle percentage risk value or range, or the lower percentage risk value or range.

20. The genetic-based biological sample analysis method of claim 18, wherein the classification as output for the user comprises the Not Likely at Increased Risk category, the classification output based upon detecting that the one or more clinically relevant allelic variant genotypes as selected for the user are associated with three or more clinically reported cases, wherein the lower percentage risk value or range is on of: (i) less than a maximum percent as defined by clinically reported cases of the respective one or more alleles associated with a not likely increased risk of developing the disease, and for a respective risk-based confidence interval or (ii) between the maximum percent as defined by clinically reported cases of the respective one or more alleles associated with the not likely increased risk of developing the disease and a minimum percent as defined the clinically reported cases of the respective one or more alleles associated with an increased risk of developing the disease, and for such respective risk-based confidence interval.

21. The genetic-based biological sample analysis method of claim 18, wherein the classification as output for the user comprises the Slightly Increased Risk category, the classification output based upon detecting that the one or more clinically relevant allelic variant genotypes as selected for the user are associated with three or more clinically reported cases, wherein the middle percentage risk value or range is at least one of: (i) more than a minimum percent as defined by clinically reported cases of the respective one or more alleles associated with an increased risk of developing the disease, and for a respective risk-based confidence interval, or (ii) between a maximum percent as defined by clinically reported cases of the respective one or more alleles associated with the not likely increased risk of developing the disease and the minimum percent as defined the clinically reported cases of the respective one or more alleles associated with an increased risk of developing the disease, and for such respective risk-based confidence interval.

22. The genetic-based biological sample analysis method of claim 18, wherein the classification as output for the user comprises the Increased Risk category, the classification output based upon detecting that the one or more clinically relevant allelic variant genotypes as selected for the user are associated with three or more clinically reported cases, wherein the upper percentage risk value or range is more than a minimum percent defined clinically reported cases of the respective one or more alleles associated with an increased risk of developing the disease, and for a respective risk-based confidence interval.

23. The genetic-based biological sample analysis method of claim 18, wherein the one or more alleles of the user are selected from one or more clinically relevant allelic variants of the serpin peptidase inhibitor class A member 1 (SERPINA1) gene, and wherein the disease is lung disease associated with alpha1-antitrypsin deficiency (A1ATD) and/or liver disease associated with alpha1-antitrypsin deficiency (A1ATD).

24. The genetic-based biological sample analysis method of claim 23, wherein the classification as output for the user comprises the Not Likely at Increased Risk category, the classification output based upon detecting at least one of: (i) the one or more clinically relevant allelic variant genotypes as selected for the user are associated with three or more clinically reported cases, the lower percentage risk value or range is less than 20 percent defining lung or liver disease occurring in less than 20 percent of clinically reported cases of the respective one or more alleles, and a respective risk-based confidence interval between zero to 80 percent, or (ii) the one or more clinically relevant allelic variant genotypes as selected for the user are associated with three or more clinically reported cases for which lung or liver disease occurred in 20 to 80 percent of clinically reported cases of the respective one or more alleles, and a respective risk-based confidence interval between zero to 80 percent.

25. The genetic-based biological sample analysis method of claim 23, wherein the classification as output for the user comprises the Slightly Increased Risk category, the classification output based upon detecting at least one of: (i) the one or more clinically relevant allelic variant genotypes as selected for the user are associated with three or more clinically reported cases, the middle percentage risk value or range is between 20 percent and 80 percent defining lung or liver disease occurring in 20 to 80 percent of clinically reported cases of the respective one or more alleles, and a respective risk-based confidence interval between 20 to 100 percent, or (ii) the one or more clinically relevant allelic variant genotypes as selected for the user are associated with three or more clinically reported cases, lung or liver disease occurred in at least 80 percent of clinically reported cases of the respective one or more alleles, and a respective risk-based confidence interval between 20 to 100 percent.

26. The genetic-based biological sample analysis method of claim 23, wherein the classification as output for the user comprises the Increased Risk category, the classification output based upon detecting the one or more clinically relevant allelic variant genotypes as selected for the user are associated with three or more clinically reported cases, the upper percentage risk value or range is greater than 80 percent defining lung or liver disease occurring in at least 80 percent of clinically reported cases of the respective one or more alleles, and a respective risk-based confidence interval greater than 80 percent.

27. The genetic-based biological sample analysis method of claim 18, wherein the lab-based genetic analysis output further comprises one or more of: (a) a quantity of clinically reported cases of the respective one or more alleles, (b) a percentage of clinically reported cases of the respective one or more alleles in which the disease occurred, and/or (c) a respective risk-based confidence interval.

28. The genetic-based biological sample analysis method of claim 18, wherein the user-specific genetic health risk determination may comprise a report selected from a preset list of report types based on the one or more clinically relevant allelic variant genotypes.

29. The genetic-based biological sample analysis method of claim 23, wherein the one or more variants of the SERPINA1 gene include one or more of: (i) PI*S; (ii) PI*Z; (iii) PI*I; (iv) PI*M procida; (v) PI*M malton; (vi) PI*S iiyama; (vii) PI*Q0 granite falls; (viii) PI*Q0 west; (ix) PI*Q0 bellingham; (x) PI*F; (xi) PI*P lowell; (xii) PI*Q0 mattawa; (xiii) PI*Q0 clayton, and (xiv) PI*M Heerlen.

30. The genetic-based biological sample analysis method of claim 18, wherein the one or more clinically relevant allelic variant genotypes comprises one or more variants of a gene, wherein the gene is a Coagulation Factor V (FV) gene, Coagulation Factor II (FII) gene, Serpin family C member 1 (SERPINC1) gene, protein S (PROS1) gene, or protein C (PROC) gene; and wherein the disease is a thrombophilia associated disease.

31. The genetic-based biological sample analysis method of claim 30, wherein the one or more clinically relevant allelic variants is selected from the group consisting of: (i) R506Q of the FV gene, (ii) FV Leiden of the FV gene, (iii) G20210A of the FII gene, (iv) A384S (G1246T) (rs121909548) of the SERPINC1 gene, (v) Intronic (rs2227589) of the SERPIN1C gene, (vi) Val30Glu (rs2227624) of the SERPIN1C gene, (vii) K196E of the PROS1 gene, (vii) K155E of the PROS1 gene, (viii) c.574_576dup/del (rs199469469) of the PROC gene, (ix) R147W of the PROC gene, (x) Arg42Cys of the PROC gene, (xi) Arg51Cys of the PROC gene, (xii) Val76Met of the PROC gene, (xiii) C 2633 G of the PROC gene, (xiv) C 2730 T of the PROC gene, (xv) G 3310 A of the PROC gene, (xvi) c.565C.T (p.Arg189Trp) of the PROC gene, (xvii) 2405C/T of the PROC gene, (xviii) 2418A/G of the PROC gene, (xix) C/T at −1654, A/G at −1641, A/T at −1476 of the PROC gene, and (xx) promoter GC haplotype of the PROC gene.

32. The genetic-based biological sample analysis method of claim 30, wherein the classification as output for the user comprises the Not Likely at Increased Risk category, the classification output based upon detecting at least one of: (i) the one or more clinically relevant allelic variant genotypes as selected for the user are associated with three or more clinically reported cases, the lower percentage risk value or range is less than 20 percent defining lung or liver disease occurring in less than 20 percent of clinically reported cases of the respective one or more alleles, and a respective risk-based confidence interval between zero to 80 percent, or (ii) the one or more clinically relevant allelic variant genotypes as selected for the user are associated with three or more clinically reported cases for which lung or liver disease occurred in 20 to 80 percent of clinically reported cases of the respective one or more alleles, and a respective risk-based confidence interval between zero to 80 percent.

33. The genetic-based biological sample analysis method of claim 30, wherein the classification as output for the user comprises the Slightly Increased Risk category, the classification output based upon detecting at least one of: (i) the one or more clinically relevant allelic variant genotypes as selected for the user are associated with three or more clinically reported cases, the middle percentage risk value or range is between 20 percent and 80 percent defining lung or liver disease occurring in 20 to 80 percent of clinically reported cases of the respective one or more alleles, and a respective risk-based confidence interval between 20 to 100 percent, or (ii) the one or more clinically relevant allelic variant genotypes as selected for the user are associated with three or more clinically reported cases, lung or liver disease occurred in at least 80 percent of clinically reported cases of the respective one or more alleles, and a respective risk-based confidence interval between 20 to 100 percent.

34. The genetic-based biological sample analysis method of claim 30, wherein the classification as output for the user comprises the Increased Risk category, the classification output based upon detecting at least one of: (i) the one or more clinically relevant allelic variant genotypes as selected for the user are associated with three or more clinically reported cases, the upper percentage risk value or range is greater than 80 percent defining lung or liver disease occurring in at least 80 percent of clinically reported cases of the respective one or more alleles, and a respective risk-based confidence interval greater than 80 percent, or (ii) the one or more clinically relevant allelic variant genotypes as selected for the user are associated with 11 or more clinically reported cases of respective other users having the one or more clinically relevant allelic variant genotypes and the disease.

35. A tangible, non-transitory computer-readable medium storing instructions for detecting a user-specific genetic health risk related to a disease, that when executed by one or more processors, cause the one or more processors to:
obtain, by one or more processors, user specific data of a user for detecting a likelihood of occurrence of a user-specific disease;
generate, by the one or more processors, a profile of the user comprising the user specific data, wherein generation of the profile causes a user test kit to be delivered to the user, wherein the user test kit is configured to collect a biological sample of the user, the biological sample comprising genomic deoxyribonucleic acid (DNA) of the user as extracted from the biological sample;
receive, at the one or more processors, lab-based genetic analysis output based on the genomic DNA of the user, wherein generation of the lab-based genetic analysis output comprises determination of one or more alleles of the user selected from one or more clinically relevant allelic variant genotypes;
output, by the one or more processors, a classification for the user, based on the lab-based genetic analysis output, by a genetic risk model, the classification defining a user-specific risk of the user to develop the disease based on the one or more alleles as selected for the user,
wherein the genetic risk model is configured to output a classification defining respective risks of respective users developing the disease,
wherein the classification as output by the genetic risk model is selected from a predetermined set of risk categories comprising: (a) an Increased Risk Category, (b) a Slightly Increased Risk Category, (c) a Not Likely at Increased Risk Category, and (d) an Unknown Risk Category,
wherein the risk categories are ordinal with respect to one another ordered based on respective percentage risk values or ranges of contracting the disease,
wherein the Increased Risk Category is assigned an upper percentage risk value or range,
wherein the Not Likely at Increased Risk Category is assigned a lower percentage risk value or range,
wherein the Slightly Increased Risk Category comprises a middle percentage risk value or range that is less than the upper percentage risk value or range but greater than the lower percentage risk value or range, and
wherein the Unknown Risk Category has an undetermined percentage risk value or range;
generate, by the one or more processors, a user-specific genetic health risk determination for the user based on the classification of the user and the lab-based genetic analysis output; and
provide, by the one or more processors, to the user the user-specific genetic health risk determination.

36. The tangible, non-transitory computer-readable medium of claim 35, wherein the classification as output for the user comprises the Unknown Risk Category, the classification output based upon detecting at least one of: (i) the one or more clinically relevant allelic variant genotypes as selected for the user are associated with two or less clinically reported cases, or (ii) the one or more clinically relevant allelic variant genotypes as selected for the user are associated with three or more clinically reported cases but the undetermined percentage risk value or range overlaps with at least one of the upper percentage risk value or range, the middle percentage risk value or range, or the lower percentage risk value or range.

37. The tangible, non-transitory computer-readable medium of claim 35, wherein the classification as output for the user comprises the Not Likely at Increased Risk category, the classification output based upon detecting that the one or more clinically relevant allelic variant genotypes as selected for the user are associated with three or more clinically reported cases, wherein the lower percentage risk value or range is one of: (i) less than a maximum percent as defined by clinically reported cases of the respective one or more alleles associated with a not likely increased risk of developing the disease, and for a respective risk-based confidence interval, or (ii) between the maximum percent as defined by clinically reported cases of the respective one or more alleles associated with the not likely increased risk of developing the disease and a minimum percent as defined the clinically reported cases of the respective one or more alleles associated with an increased risk of developing the disease, and for such respective risk-based confidence interval.

38. The tangible, non-transitory computer-readable medium of claim 35, wherein the classification as output for the user comprises the Slightly Increased Risk category, the classification output based upon detecting that the one or more clinically relevant allelic variant genotypes as selected for the user are associated with three or more clinically reported cases, wherein the middle percentage risk value or range is at least one of: (i) more than a minimum percent as defined by clinically reported cases of the respective one or more alleles associated with an increased risk of developing the disease, and for a respective risk-based confidence interval, or (ii) between a maximum percent as defined by clinically reported cases of the respective one or more alleles associated with the not likely increased risk of developing the disease and the minimum percent as defined the clinically reported cases of the respective one or more alleles associated with an increased risk of developing the disease, and for such respective risk-based confidence interval.

39. The tangible, non-transitory computer-readable medium of claim 35, wherein the classification as output for the user comprises the Increased Risk category, the classification output based upon detecting that the one or more clinically relevant allelic variant genotypes as selected for the user are associated with three or more clinically reported cases, wherein the upper percentage risk value or range is more than a minimum percent defined clinically reported cases of the respective one or more alleles associated with an increased risk of developing the disease, and for a respective risk-based confidence interval.

40. The tangible, non-transitory computer-readable medium of claim 35, wherein the one or more alleles of the user are selected from one or more clinically relevant allelic variants of the serpin peptidase inhibitor class A member 1 (SERPINA1) gene, and wherein the disease is lung disease associated with alpha1-antitrypsin deficiency (A1ATD) and/or liver disease associated with alpha1-antitrypsin deficiency (A1ATD).

41. The tangible, non-transitory computer-readable medium of claim 40, wherein the classification as output for the user comprises the Not Likely at Increased Risk category, the classification output based upon detecting at least one of: (i) the one or more clinically relevant allelic variant genotypes as selected for the user are associated with three or more clinically reported cases, the lower percentage risk value or range is less than 20 percent defining lung or liver disease occurring in less than 20 percent of clinically reported cases of the respective one or more alleles, and a respective risk-based confidence interval between zero to 80 percent, or (ii) the one or more clinically relevant allelic variant genotypes as selected for the user are associated with three or more clinically reported cases for which lung or liver disease occurred in 20 to 80 percent of clinically reported cases of the respective one or more alleles, and a respective risk-based confidence interval between zero to 80 percent.

42. The tangible, non-transitory computer-readable medium of claim 40, wherein the classification as output for the user comprises the Slightly Increased Risk category, the classification output based upon detecting at least one of: (i) the one or more clinically relevant allelic variant genotypes as selected for the user are associated with three or more clinically reported cases, the middle percentage risk value or range is between 20 percent and 80 percent defining lung or liver disease occurring in 20 to 80 percent of clinically reported cases of the respective one or more alleles, and a respective risk-based confidence interval between 20 to 100 percent, or (ii) the one or more clinically relevant allelic variant genotypes as selected for the user are associated with three or more clinically reported cases, lung or liver disease occurred in at least 80 percent of clinically reported cases of the respective one or more alleles, and a respective risk-based confidence interval between 20 to 100 percent.

43. The tangible, non-transitory computer-readable medium of claim 40, wherein the classification as output for the user comprises the Increased Risk category, the classification output based upon detecting the one or more clinically relevant allelic variant genotypes as selected for the user are associated with three or more clinically reported cases, the upper percentage risk value or range is greater than 80 percent defining lung or liver disease occurring in at least 80 percent of clinically reported cases of the respective one or more alleles, and a respective risk-based confidence interval greater than 80 percent.

44. The tangible, non-transitory computer-readable medium of claim 35, wherein the lab-based genetic analysis output further comprises one or more of: (a) a quantity of clinically reported cases of the respective one or more alleles, (b) a percentage of clinically reported cases of the respective one or more alleles in which the disease occurred, and/or (c) a respective risk-based confidence interval.

45. The tangible, non-transitory computer-readable medium of claim 35, wherein the user-specific genetic health risk determination may comprise a report selected from a preset list of report types based on the one or more clinically relevant allelic variant genotypes.

46. The tangible, non-transitory computer-readable medium of claim 40, wherein the one or more variants of the SERPINA1 gene include one or more of: (i) PI*S; (ii) PI*Z; (iii) PI*I; (iv) PI*M procida; (v) PI*M malton; (vi) PI*S iiyama; (vii) PI*Q0 granite falls; (viii) PI*Q0 west; (ix) PI*Q0 bellingham; (x) PI*F; (xi) PI*P lowell; (xii) PI*Q0 mattawa; (xiii) PI*Q0 clayton, and (xiv) PI*M Heerlen.

47. The tangible, non-transitory computer-readable medium of claim 35, wherein the one or more clinically relevant allelic variant genotypes comprises one or more variants of a gene, wherein the gene is a Coagulation Factor V (FV) gene, Coagulation Factor II (FII) gene, Serpin family C member 1 (SERPINC1) gene, protein S (PROS1) gene, or protein C (PROC) gene; and wherein the disease is a thrombophilia associated disease.

48. The tangible, non-transitory computer-readable medium of claim 47, wherein the one or more clinically relevant allelic variants is selected from the group consisting of: (i) R506Q of the FV gene, (ii) FV Leiden of the FV gene, (iii) G20210A of the FII gene, (iv) A384S (G1246T) (rs121909548) of the SERPINC1 gene, (v) Intronic (rs2227589) of the SERPIN1C gene, (vi) Val30Glu (rs2227624) of the SERPIN1C gene, (vii) K196E of the PROS1 gene, (vii) K155E of the PROS1 gene, (viii) c.574_576dup/del (rs199469469) of the PROC gene, (ix) R147W of the PROC gene, (x) Arg42Cys of the PROC gene, (xi) Arg51Cys of the PROC gene, (xii) Val76Met of the PROC gene, (xiii) C 2633 G of the PROC gene, (xiv) C 2730 T of the PROC gene, (xv) G 3310 A of the PROC gene, (xvi) c.565C.T (p.Arg189Trp) of the PROC gene, (xvii) 2405C/T of the PROC gene, (xviii) 2418A/G of the PROC gene, (xix) C/T at −1654, A/G at −1641, A/T at −1476 of the PROC gene, and (xx) promoter GC haplotype of the PROC gene.

49. The tangible, non-transitory computer-readable medium of claim 47, wherein the classification as output for the user comprises the Not Likely at Increased Risk category, the classification output based upon detecting at least one of: (i) the one or more clinically relevant allelic variant genotypes as selected for the user are associated with three or more clinically reported cases, the lower percentage risk value or range is less than 20 percent defining lung or liver disease occurring in less than 20 percent of clinically reported cases of the respective one or more alleles, and a respective risk-based confidence interval between zero to 80 percent, or (ii) the one or more clinically relevant allelic variant genotypes as selected for the user are associated with three or more clinically reported cases for which lung or liver disease occurred in 20 to 80 percent of clinically reported cases of the respective one or more alleles, and a respective risk-based confidence interval between zero to 80 percent.

50. The tangible, non-transitory computer-readable medium of claim 47, wherein the classification as output for the user comprises the Slightly Increased Risk category, the classification output based upon detecting at least one of: (i) the one or more clinically relevant allelic variant genotypes as selected for the user are associated with three or more clinically reported cases, the middle percentage risk value or range is between 20 percent and 80 percent defining lung or liver disease occurring in 20 to 80 percent of clinically reported cases of the respective one or more alleles, and a respective risk-based confidence interval between 20 to 100 percent, or (ii) the one or more clinically relevant allelic variant genotypes as selected for the user are associated with three or more clinically reported cases, lung or liver disease occurred in at least 80 percent of clinically reported cases of the respective one or more alleles, and a respective risk-based confidence interval between 20 to 100 percent.

51. The tangible, non-transitory computer-readable medium of claim 47, wherein the classification as output for the user comprises the Increased Risk category, the classification output based upon detecting at least one of: (i) the one or more clinically relevant allelic variant genotypes as selected for the user are associated with three or more clinically reported cases, the upper percentage risk value or range is greater than 80 percent defining lung or liver disease occurring in at least 80 percent of clinically reported cases of the respective one or more alleles, and a respective risk-based confidence interval greater than 80 percent, or (ii) the one or more clinically relevant allelic variant genotypes as selected for the user are associated with 11 or more clinically reported cases of respective other users having the one or more clinically relevant allelic variant genotypes and the disease.
